(19) United States
(12) Patent Application Publication
(10) Pub. No.: US 2026/0083571 A1
(43) Pub. Date: Mar. 26, 2026

(54) EXPANDABLE AND CONFORMING SPINAL IMPLANT

(71) Applicant: Nexus Spine, LLC, Salt Lake City, UT (US)

(72) Inventors: David T. Hawkes, Salt Lake City, UT (US); Peter A. Halverson, Salt Lake City, UT (US); Justin G. Heap, Salt Lake City, UT (US)

(21) Appl. No.: 19/409,300

(22) Filed: Dec. 9, 2025

Related U.S. Application Data
(63) Continuation of application No. 18/963,046, filed on Nov. 27, 2024.
(60) Provisional application No. 63/603,558, filed on Nov. 28, 2023.
(60) Provisional application No. 63/550,491, filed on Feb. 6, 2024.
(60) Provisional application No. 63/674,445, filed on Jul. 23, 2024.

(51) Int. Cl. A61F 2/44

(52) U.S. Cl. CPC A61F 2/442; A61F 2210/00; A61F 2250/0007

(57) ABSTRACT

Systems and methods of providing one or more conforming interbody implants, implants with one or more conforming regions, or related implants are described. According to some implementations, an interbody implant includes a first surface configured to be disposed adjacent to a first bony endplate, and a second surface configured to be disposed adjacent to a second body endplate, with a height of the interbody implant extending between the first surface and the second surface. According to some implementations, the interbody implant is configured to be selectively disposed in an insertion configuration and a deployed configuration. In some cases, the height of the interbody implant is greater in the deployed configuration than in the insertion configuration. Moreover, in some cases, the first surface is configured to conform to a topology of the first bony endplate, and the second surface is configured to conform to a topology of the second bony endplate.

### RELATED APPLICATIONS AND PRIORITY CLAIMS

This application is a continuation of and claims priority to U.S. patent application Ser. No. 18/963,046, entitled EXPANDABLE AND CONFORMING SPINAL IMPLANT, filed Nov. 27, 2024 (Attorney Docket No. 23845.200), which claims priority to: (a) U.S. Provisional Patent Application No. 63/603,558, entitled EXPANDABLE AND CONFORMING SPINAL IMPLANT, filed Nov. 28, 2023 (Attorney Docket No. 23845.163); (b) U.S. Provisional Patent Application No. 63/674,445, entitled IMPLANT WITH CONFORMING REGION, filed Jul. 23, 2024 (Attorney Docket No. 23845.181); and (c) U.S. Provisional Patent Application No. 63/550,491, entitled SYSTEMS AND METHODS FOR PROVIDING AND DEPLOYING INTERBODY IMPLANTS, filed Feb. 6, 2024 (Attorney Docket No. 23845.177). The contents of each of the aforementioned applications are incorporated herein by reference in their entireties.

### FIELD

The present disclosure relates to medical devices, and more particularly to spinal or other orthopedic implants.

### BACKGROUND

Traditionally, it is difficult to properly place a spinal interbody implant for a variety of reasons. For example, the interbody space to be filled is typically larger than the surgical access window allowed by surrounding anatomical structures.

Additionally, a goal of implanting an interbody device is oftentimes to increase a height of an interverbal space. However, it can be difficult to assess which interbody height would best fill the interbody space. Therefore, the selected interbody implant may be too tall, thereby applying excessive, painful, and traumatic force to spinal vertebrae and surrounding tissues, or the interbody implant may be too short, thereby presenting a risk of expulsion, migration, and painful instability.

Additionally, some traditional implants do not conform well to natural structures, such as bony regions. In this regard, they are typically too rigid to take on the shape of the mating anatomy. This can result in point loading in some regions and voids in other regions. For example, some traditional spinal implants do not always conform well to a natural bony endplate of a spinal segment of a patient. Such conformity failures can result in voids or gaps that are slow to backfill, localized regions of high contact stress that lead to bone loss and subsidence, and other serious issues that can cause instability, loss of function, pain, and decreased quality of life for patients.

Some implants create regions of hyperloading, or excessive strain, which can result in micro or macro fractures. Similarly, some implants create regions of hypoloading, which can lead to bone atrophy and prevent bone healing. In either case, these shortcomings can lead to a patient experiencing large amounts of pain, reduced physical capacity, and other major problems.

Thus, while techniques currently exist that are used to provide spinal implants, challenges still exist, including those listed above. Accordingly, it would be an improvement in the art to augment or even replace current techniques with other techniques.

### SUMMARY

When a spinal implant is applied, implant-to-bone contact can be critical for the healthy formation of bone. Such contact can impact many factors, such as stability, contact stress, rate of bone formation, mechanical stimulus of bone for continued health, expulsion, and other factors. In prior systems, implants are often selected that are too tall. This can cause numerous problems, such as subsidence due to excessive contact force, endplate violation during impaction, and other problems. Accordingly, systems and methods for providing an expandable and conforming spinal implant are disclosed herein. In some implementations, the described expandable and conforming spinal implant avoids the problems present in some prior systems.

As mentioned above, some implementations of the implant are configured to be expandable. In this regard, some iterations of the implant have an insertion configuration, in which the implant has a lower profile and is configured to be inserted into an interbody space, and a deployed configuration, in which at least a portion of the height of the implant is increased to provide the necessary support for the corresponding bony endplates of a patient's spine. Moreover, some implementations of the implant are configured to be conforming, allowing a caudal or cranial surface to conform to one or more of the specific contours and topology of an adjacent bony endplate.

Some implementations of the implant are configured to be conforming with a contact force limit. In this regard, the contact force limit can be provided such that the incidence of high-pressure contact points of the implant on the endplate, as well as substantial gaps between the implant and the endplate, are reduced to levels below the fracture limit of a vertebral endplate, or trabecular bone, or other relevant anatomical structures, such as ligaments, facets capsules, etc. Thus, in some implementations, a more even and consistent support structure for the endplate is provided.

In some implementations, the implant has a modular structure that includes multiple columns, which, in some cases, include one or more fixed columns and one or more expanding columns. Together, fixed columns and expanding columns can provide the implant with the ability to expand to the deployed configuration, conform with endplate topology, and still provide stability and structural support.

Where the implant includes expanding columns, some implementations of the expanding columns are equipped with adjustable components, allowing them to increase in height to expand the implant and apply a controlled load to the bony endplates. In some cases, these expanding columns include one or more regions with varying characteristics, such as stiffness, porosity, and material properties-enabling them to provide tailored support and adjustability. For instance, some instances of outer regions include higher stiffness for structural support, while some instances of inner regions include one or more expandable components to facilitate deployment (e.g., expansion) of the implant.

Where the implant includes fixed columns, some implementations of the fixed columns provide consistent structural support. In some cases, they are used alongside expanding columns to stabilize the implant after deployment. For example, expanding columns are (in some cases) coupled to fixed columns to transfer the load of the endplates through the fixed columns.

Although some embodiments of the fixed columns are rigid, some other embodiments of the fixed columns are not; rather, they are fixed in a relative sense when compared to the expanding columns. Still, some embodiments of the fixed columns are compressible, with a stiffness at or below the stiffness of the surrounding bone to allow for bone loading. Also, some embodiments of the fixed columns are porous.

While the expanding columns can have any suitable characteristic, in some embodiments, they are configured to limit the contact pressure on the surrounding bone to less than 1 GPa, or even less than 0.1 GPa. Although limiting contact pressure can serve any suitable purpose, in some cases, the intent of limiting the contact pressure is to prevent bone fracture or bone damage.

According to some implementations of the systems and methods disclosed herein, an implant with one or more conforming regions is provided. The conforming region can include any component or configuration that allows the implant to conform to a variety of surfaces, such as one or more different spinal segment endplates having different topologies. For example, the conforming region can include one or more porous regions, which in turn may include one or more springs, sponges, filaments, flexures, lattices, scaffolds, grids, seals, foams, cushions, layers of any of the foregoing, or any other strands, or any other suitable components capable of conforming to an uneven surface (or a surface that is not completely planar). In some cases, the conforming region is configured to apply an even load across an uneven surface (e.g., a bony endplate of a spinal segment).

Although the conforming region can be provided in any suitable manner, some implementations of the implant include one or more load-bearing regions (e.g., at least a first load-bearing region and a second load-bearing region, although a third load-bearing region, a fourth load-bearing region, and any additional number of load-bearing regions can also be included). In some iterations, one or more load-bearing regions form the conforming region (e.g., by having a stiffness that is low enough to allow for conformity across an uneven surface), while one or more other load-bearing regions form a more rigid (less conforming) portion of the implant configured to provide desired stiffness (such that the entirety of the implant is not overly conforming, which could lead to hypoloading and failure of the implant to remain in its proper position).

Where the implant includes multiple load-bearing regions, the load-bearing regions can be arranged in series, in parallel, in a combination of series and parallel arrangements, or in any other suitable manner. In some cases, at least two load-bearing regions are arranged in parallel such that load can be placed on a first load-bearing region without placing load on a second load-bearing region. Further, in some cases, the first load-bearing region is positioned closer to a point of contact than is the second load-bearing region (e.g., thereby allowing the first load-bearing region to conform to an uneven surface before load is placed on the second load-bearing region).

In some implementations, the implant is “double-sided” such as by having a first conforming region disposed at (or proximate) a rostral face of the implant as well as a second conforming region disposed at (or proximate) a caudal face of the implant. Again, this can be accomplished through any suitable arrangement, but in some cases this is done through a plurality of load-bearing regions (e.g., with one or more compliant load-bearing regions being positioned nearer points of contact than stiffer load-bearing regions).

Some iterations of the implant include one or more porous structures. The porous structure can include any suitable structure having one or more gaps, spaces, air pockets, voids, orifices, cavities, hollows, or any other pores capable of promoting bone growth. In some cases, the configuration of the porous structure at least partially gives rise to one or more of the load-bearing regions. For example, in some cases the porous structure includes one or more coils, strips, filaments, rods, fibers, sponge-like structures, or other strands configured to form spring-like structures that give rise to the various stiffnesses of the load-bearing regions.

Some implementations of the implant include one or more additional implant components for increasing the usefulness, efficacy, safety, or other beneficial effects of the device. For example, some iterations include one or more primary fixation mechanisms for securing the implant in place, and some iterations include one or more passages (such as cavities, conduits, depressions, holes, slots, receptacles, or other orifices) for receiving one or more materials or tools, housing components (such as one or more anchors), adjusting one or more parameters (e.g., stiffness) of one or more portions of the implant, or otherwise augmenting or adjusting the implant's characteristics.

Many other iterations and implementations are possible, and the features and benefits of the instant systems and methods are explained and illustrated in much greater detail in the following disclosure and in the corresponding figures.

### DESCRIPTION OF THE FIGURES

The objects and features of the present disclosure will become more fully apparent from the following description and appended claims, taken in conjunction with the accompanying figures. Understanding that these figures depict only some embodiments of the disclosed systems and methods and are, therefore, not to be considered limiting in scope, the systems and methods will be described and explained with additional specificity and detail through the use of the accompanying figures in which:

FIG. 1A shows an X-ray image of a patient's spine with an interbody implant disposed therein, in accordance with an illustrative example of patient anatomy;

FIG. 1B shows an X-ray image of a patient's spine with a collapsed disc space, in accordance with another illustrative example of patient anatomy;

FIG. 1C shows an image of a bony endplate of a spinal segment of a patient to illustrate a topology thereof, in accordance with another illustrative example of patient anatomy;

FIG. 1D shows an X-ray image of a patient's spine to illustrate how rigid body implants can create gaps between implant endplates and bony endplates of the patient's spinal segments, in accordance with another illustrative example of patient anatomy;

FIG. 2A shows a side perspective view of an expandable and conforming interbody spacer, in accordance with a representative embodiment of the present systems and methods;

FIG. 2B shows a top perspective view of the expandable and conforming interbody spacer, in accordance with some representative embodiments;

FIGS. 3A and 3B each show a front elevation view of the expandable and conforming interbody spacer implant, in each case abutting a bony endplate of a spinal segment of a patient, in accordance with some representative embodiments;

FIGS. 3C and 3D each show a cross-sectional elevation view of the expandable and conforming interbody spacer implant, in each case abutting a bony endplate of a spinal segment of a patient, in accordance with some representative embodiments;

FIG. 3E shows a rear elevation view of the expandable and conforming interbody spacer implant abutting a bony endplate of a spinal segment of a patient, in accordance with some representative embodiments;

FIG. 4A shows some expanding columns as may be used in the expandable and conforming interbody spacer implant, in accordance with some representative embodiments;

FIGS. 4B and 4C each show some columns, including expanding columns and fixed columns, as may be used in the expandable and conforming interbody spacer implant, in accordance with some representative embodiments;

FIGS. 5A-5E show diagrams illustrating various configurations of columns, as may be used in the expandable and conforming interbody spacer implant, in accordance with some representative embodiments;

FIG. 6A shows a side elevation view of an example coupling mechanism for selectively coupling one column to another column, in accordance with some representative embodiments;

FIG. 6B shows a plan view of an example coupling mechanism for selectively coupling one column to another column, in accordance with some representative embodiments;

FIGS. 7 and 8 show plan views of examples of column arrangements for columns of interbody spacer implants, in accordance with some representative embodiments;

FIGS. 9 and 10 each show a front perspective view of the expandable and conforming interbody spacer implants, according to some representative embodiments;

FIGS. 11A-11E show cross-sectional views depicting natural variance in endplate topology of human spinal segments;

FIG. 12 shows a plan view of a bony endplate of a spinal segment, divided into regions that align with regions of a spinal implant in accordance with some representative embodiments;

FIG. 13 shows an elevation view of a spinal implant that is not configured to conform with endplate topology, as taught in prior art;

FIG. 14 shows a cross-sectional elevation view of an endplate of a spinal implant having a conforming region configured to conform with endplate topology, in accordance with some embodiments of the disclosed systems and methods;

FIG. 15 shows a cross-sectional elevation view of the endplate of the spinal implant of FIG. 4 under load, in accordance with some representative embodiments;

FIG. 16 shows an elevation view of a spinal implant having one or more conforming regions, in accordance with some representative embodiments;

FIG. 17 shows a front perspective view of a spinal implant having one or more conforming regions, in accordance with some representative embodiments;

FIG. 18 shows a top perspective view of a spinal implant having one or more conforming regions, in accordance with some representative embodiments;

FIG. 19 shows a front perspective view of a spinal implant having a conforming region, with a primary fixation mechanism retracted, in accordance with some representative embodiments;

FIG. 20 shows a front perspective view of the spinal implant of FIG. 9 having one or more conforming regions, with the primary fixation mechanism deployed, in accordance with some representative embodiments; and

FIG. 21 shows a close-up view of a portion of a spinal implant having one or more conforming regions, in accordance with some representative embodiments.

### DETAILED DESCRIPTION

A description of embodiments will now be given with reference to the figures. It is expected that the present systems and methods may take many other forms and shapes. Hence, the following disclosure is intended to be illustrative and not limiting, and the scope of the disclosure should be determined by reference to the appended claims.

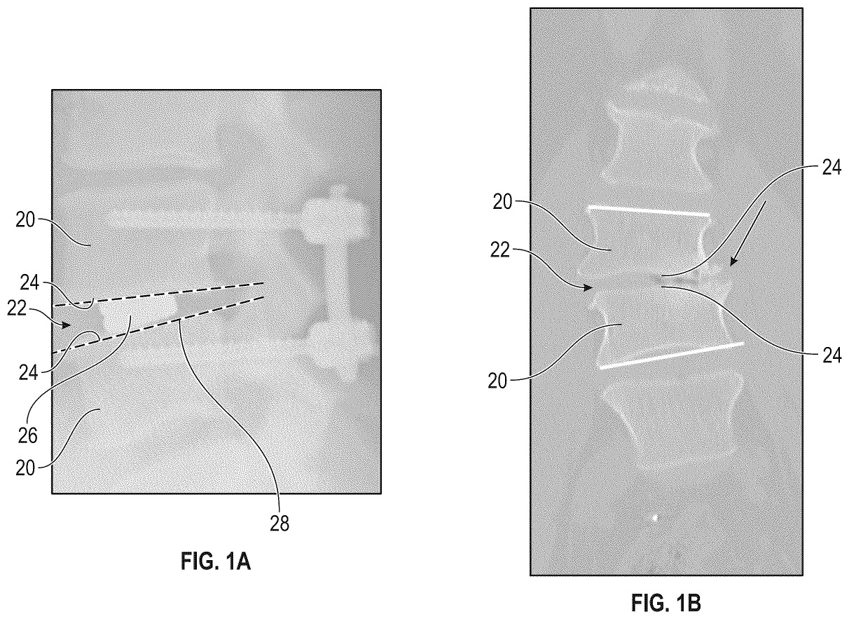

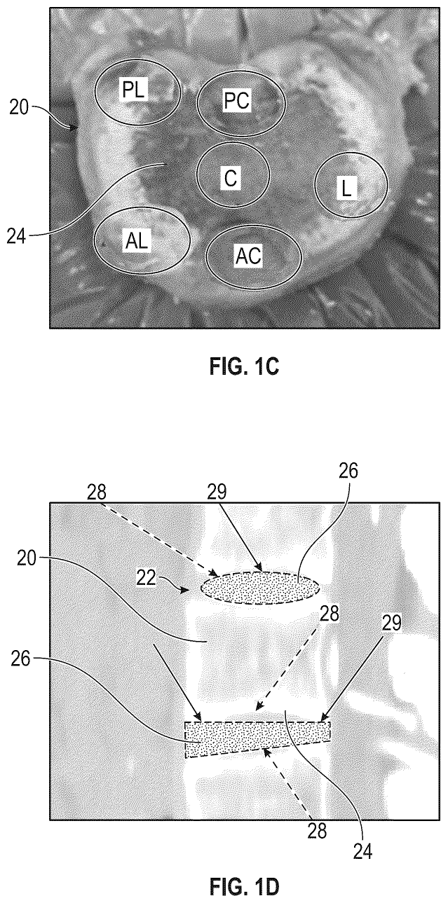

Some embodiments will now be described with reference generally to FIGS. 1A-10. Interbody implants (also known as interbody spacers) are generally configured to be inserted between two vertebral bodies 20 of a patient's spine, into an intervertebral space 22. The implants are typically further configured to contact the bony endplates 24 of the adjacent vertebral bodies. For example, FIG. 1A shows a rigid implant 26 disposed in an intervertebral space 22 between the bony endplates 24 of adjacent spinal vertebrae 20.

Unfortunately, in some cases, an intervertebral space 22 can collapse (e.g., the endplates can shift toward each other and even, in some cases, contact each other). For example, FIG. 1B shows a pair of vertebral bodies 20 that have shifted toward each other, their endplates 24 contacting each other, thereby drastically shrinking the intervertebral space. When this happens, it can be extremely difficult to insert an implant, particularly an implant that is tall enough to provide the proper spacing between the vertebral bodies.

Moreover, some traditional spinal interbody implants (e.g., a rigid implant 26) do not always conform well to a patient's natural endplates 24 of the patient's vertebrae 20. Such conformity failures can result in voids or gaps that are slow to backfill, localized regions of high contact stress that lead to bone loss and subsidence, and other serious issues that can cause loss of function, pain, and decreased quality of life for patients. To further explain the foregoing, bony endplates of vertebral bodies typically each have their own unique topology. Such topology can vary greatly between endplates across patients and even across spinal segments of a single patient. For example, the topology of a bony endplate often forms a bowl shape, a plate shape, a parabolic dish shape, or another curved configuration. Such curvature, however, can vary substantially from one endplate to another and even within a single endplate. In this regard, the topology of a given endplate generally has a depth (or a distance from a most raised portion to a most depressed portion of the endplate), which may often range from anywhere between 0.01 mm and 4 mm (or even up to 10 mm, or any subrange between 0.01 mm and 10 mm). By way of example, a cranial (upper) endplate may have a depth of about 3.6 mm, while the topology of a caudal (lower) endplate may have a depth of about 2.6 mm. Meanwhile, another vertebra (e.g., a vertebra of another patient or another vertebra within the same patient) may have a caudal endplate with a depth of about 0.4 mm, and a rostral endplate with a depth of about 4.1 mm. Many other depths and combinations are also possible.

In some cases, in addition to having a particular depth, the topology of an endplate 24 fits into a particular topological category, such as shallow-shallow, shallow-flat, deep-shallow, deep-flat, or one or more other categories. In some cases, the topology does not necessarily fit well into a particular category. Moreover, the topology can also have regional variations. Generally speaking, a position on a topology of an endplate can be identified with the terms central (toward a midline of the endplate), lateral (away from the midline), posterior (toward a back of the patient), and anterior (toward a front of the patient). Thus, specific regions can be identified, as shown in FIG. 1C, such as central (C), lateral (L), posterior-central or (PC), anterior-central (AC), posterior-lateral (PL), or anterior-lateral (AL). Often, each region has its own natural variations (e.g., different depths, surface features, shapes, or other variations), thereby leading to a highly diverse possible range of topologies. Generally, the vertebral endplates are lenticular and the cranial side of the intervertebral space has more concavity than does the caudal side. Moreover, the amount of concavity can vary from one vertebral level to the next.

In some cases, a surgeon may need to revise a previous surgery, remove a previous implant, and repair damage to the bone and surrounding anatomy. For example, a previous implant could have subsided into the vertebral bodies and created a void in the bone that would need to be filled. In some such cases, the described conforming implant can be configured to fill the bone damage and repair the iatrogenic defect. Moreover, in such a case, additional volume and displacement of the expanding and fixed structures to conform to the void can, in some embodiments, be advantageous.

In short, each endplate 24 tends to have its own unique topology, with its own particular microscopic and macroscopic variations. Accordingly, many traditional spinal implants-which generally have no ability (or only limited ability) to conform to topological variations-often do not fit well with different endplates. Indeed, as shown in FIG. 1D, rigid or non-conforming implants 26 tend (in some cases) to create regions of hyperloading, or excessive strain, such as a contact point 29 where a portion of an endplate 20 directly contacts the rigid implant. Hyperloading can result in micro or macro fractures in the patient's spinal segments, causing pain and potentially leading to complications requiring future surgery. Similarly, some existing implants also tend to create regions of hypoloading, such as where there are gaps 28 between the implant and the body endplate (e.g., due to the endplate's depth 24). Hypoloading can lead to bone atrophy, instability of the implant, or other issues. In short, many traditional implants do not create even loading across spinal endplates having varying topologies. These shortcomings can lead to a patient experiencing large amounts of pain, reduced physical capacity, and other major problems.

Furthermore, healthy bone strength is generally no more than 8-14 N/mm², and osteopenic bone strength is typically even less than that. Indeed, in some cases, healthy vertebral bone stiffness is typically between 100 MPa and 600 Mpa (N/mm²). As a result, an implant of appropriate stiffness could potentially exceed the failure load of the endplate after ˜0.1 mm of expansion. Accordingly, it would, in some embodiments, be desirable to have an implant that can expand at a different, lower stiffness than would be ideal for its routine, daily loading.

Furthermore, by having the implant conform to the shape, or near the shape, of the bone, the implant is, in some embodiments, more capable of being retained within the interbody space by the natural endplate (e.g., as a convex shape in a concave shape is better retained than a flat shape on a concave shape).

To address these and other problems, some embodiments of the systems and methods described herein include one or more expandable and conforming interbody spacer implants 30 (sometimes referred to herein simply as implants or the implant).

According to some embodiments, the implant 30 is configured to expand (e.g., to increase a height of the implant extending between a first bony endplate 24 of a first vertebral body 20 and a second bony endplate of a second vertebral body). Accordingly, some embodiments of the implant are configured to be inserted into an intervertebral space 22 while the implant is in an insertion configuration. In some cases, the implant is condensed, compressed, or low-profile, or otherwise has a smaller height when it is in the insertion configuration such that it can be more easily inserted into an interbody space (for example, an interbody space that has collapsed, or any other suitable interbody space). In some embodiments, the implant further has a deployed configuration in which the height of the implant is greater than the height of the implant in the insertion configuration. Accordingly, in some cases, the implant is configured to expand when it is toggled (or otherwise adjusted) from the insertion configuration to the deployed configuration—thus allowing it to be inserted more easily, yet to still adopt the proper height to provide the needed spacing and support for the adjacent vertebral bodies 20. In some embodiments, the implant further has a deployed configuration in which the height of the implant is less than the height of the implant in the insertion configuration; that is to say that the deployed configuration can be force limited, and so it is possible that when the implant is toggled from the insertion configuration to the deployed configuration, the implant could shrink in height if the loads within the space are too high for the implant to grow in height. The interbody spacer implant can have any suitable components or characteristics that allow it to toggle (or otherwise adjust) from an insertion configuration to a deployed configuration (i.e., to be deployed), as discussed in more detail below.

Where the implant 30 is configured to expand, it can be configured to expand any suitable amount. For example, according to some embodiments, a height of one or more portions of the implant (or a height of the implant overall) is configured to expand (e.g., from an insertion configuration to a deployed configuration) by anywhere from 1% to 500%, or any subrange thereof (e.g., between 1% and 100%, 5% and 90%, 10% and 85%, 20% and 80%, 25% and 75%, or any other suitable subrange).

According to some embodiments, the implant 30 is configured to conform to one or more bony endplates 24. Thus, in some cases, the implant is configured to eliminate one or more gaps between a surface of the implant and a bony endplate, provide a desired level of support across an uneven topology (or multiple different kinds of topologies) of a bony endplate, adopt a surface configuration that corresponds to the topology of a bony endplate, or otherwise conform to a bony endplate to avoid hyperloading or hypoloading. In some cases, the implant is configured to conform (e.g., adopt a conforming configuration) as it is deployed. Thus, some embodiments of the deployed configuration are also configured to conform to the topology of one or more bony endplates. In this regard, the interbody spacer implant can have any suitable components or characteristics that allow it to conform to one or more bony endplates.

Where the implant 30 is configured to conform to one or more bony endplates 24, the implant can conform to any suitable degree. For example, in some embodiments, a first portion of the implant expands substantially more than a second portion of the implant when the implant is deployed. In some cases, a first portion of the implant is configured to expand up to 200× more or less than a second portion of the implant when the implant is deployed (e.g., 150×, 100×, 75×, 50×, 25×, 10×, 5×, 2×, or any other suitable difference in expansion). In this regard, it is noted that, in some cases, the need for the most expansion is nearest to the center of the vertebral endplate. Accordingly, in some embodiments, a portion of the implant nearest its center is configured to expand more. Alternatively, in some embodiments, a bone defect may require an implant to be configured to expand most in the area of the bone defect. Thus, some embodiments of the described implant are tailored to the specific characteristics of one or more bones that are to be adjacent to the implant once it is properly seated in the patient.

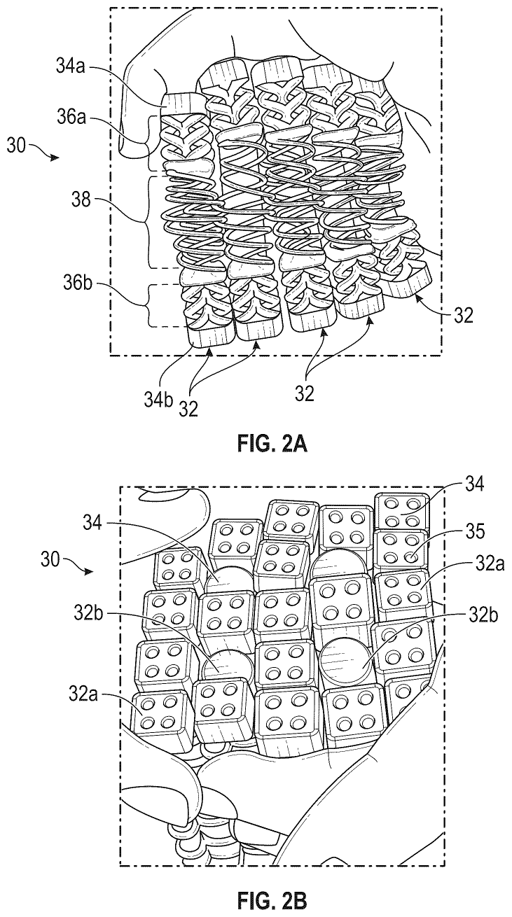

As mentioned above, the implant 30 can be configured to expand and conform in any suitable manner, but some embodiments of the implant include a plurality of components, which can include expanding components and fixed components (and any other type of components). These components can include any suitable components, such as one or more sheets, walls, rods, springs, frames, blocks, filaments, strands, coils, scaffolds, waveforms, or other components. For example, in some embodiments the components include one or more columns 32 configured to extend between a first bony endplate 24 of a first vertebral body 20 and a second bony endplate 24 of a second vertebral body 20. Columns (e.g., generally columnar components, in accordance with some embodiments, or any other suitable structures) may be particularly useful for some embodiments of the instant systems and methods. Accordingly, this disclosure generally describes the characteristics of the components with respect to columns. That said, for the purposes of this disclosure, references to columns can be substituted for references to other components, such as cubic, disc-like, sheet-like, prismatic, elliptical, elongated, conical, frustoconical, columnar, polygonal, symmetrical, asymmetrical, wave-like, strand-like, arch-like, or otherwise-shaped components, and the features described with respect to columns can be applied to such otherwise-shaped components. By way of non-limiting illustration, FIGS. 2A and 2B show some embodiments of the implant 30 having a plurality of columns 32, including one or more expanding columns 32a and fixed columns 32b.

The implant 30 can include any suitable number of columns 32 (or other components), such as anywhere from 2 to 1,000, or any subrange thereof (e.g., 5-50, 8-30, 10-20, 11-13, or any other suitable number).

Where the implant 30 includes one or more columns 32, the columns can be formed of or otherwise include any suitable material or materials. For example, some embodiments include one or more types of metal (e.g., titanium, titanium alloy, steel (e.g., stainless steel or other steel), tantalum, cobalt, chromium, magnesium, platinum, copper, aluminum, silicon nitride, ceramic, or any other suitable biocompatible metal or metal alloy), polymer material (e.g., polyethylene, polyamides, polyurethane, polycarbonate, polypropylene, polystyrene, polyvinyl chloride (PVC), acrylonitrile butadiene styrene (ABS), polylactic acid (PLA), polyetheretherketone (PEEK), polyaryletherketone (PEAK), or any other biocompatible polymer material), glass, plastic, carbon fiber, wood, fabric, ceramic, fiberglass, or any other material. By way of non-limiting illustration, some embodiments include titanium or one or more other metal materials, as such material selection can impart desirable strength characteristics to the implant. Moreover, some embodiments of the columns comprise silicon nitride.

Where the implant 30 comprises more than one column 32, the various columns can all comprise the same, different, or any suitable combination of material or materials. Indeed, in some embodiments, a first column is formed of or otherwise includes a first material or combination of materials, and a second column is formed of or otherwise includes a second material or combination of materials. In some cases, the first material or combination of materials is the same as the second material or combination of materials, but in some cases it is different. Similarly, a third column can include a third material (or combination of materials), which can be the same as or a different material from the first or second materials (or combinations), as can a fourth column, a fifth column, and any other suitable number of additional columns. Moreover, in accordance with some embodiments, selecting materials of varying moduli can enhance the specific function of each specific column (e.g., fixed columns 32b could be of a stiffer material and expanding columns 32a could be of a more flexible material). Additionally, in some embodiments, one or more materials can be selected for percent elongation at yield to enhance any suitable type of performance, such as fatigue resistance.

The columns 32 can also include any suitable configuration allowing them to load against (e.g., apply pressure, directly or indirectly, to) a spinal segment endplate 24. In some embodiments, the configuration of the columns (alone or in combination) allows the implant 30 (or one or more portions thereof) to conform to a variety of endplate topologies and to place a satisfactory load on a variety of endplates (without hypoloading or hyperloading the endplate or portions thereof) despite topological variations of such endplates. By way of example, some embodiments of the columns include one or more fixed components, stiff components, solid components, hollow components, expanding components, porous regions, frame regions, spring regions, resilient regions, rigid regions, fibrous regions, filamentous regions, foam regions, spongy regions, inflatable regions, flexures, grids, seal regions, cushion regions, sheets, curves, lattices, scaffolds, honeycombs, coil packs, or any other suitable configuration of one or more features that are configured to impart particular stiffnesses, strengths, or any other suitable attributes to the columns. Some examples of specific configurations of some suitable columns are provided in a later portion of this description below.

The columns 32 of the implant 30 can have any suitable stiffnesses for conforming to an endplate 24 and exerting a suitable load on the endplate. According to some embodiments, one or more of the columns is configured to have a stiffness between 10 MPa and 300 GPa (or any subrange thereof), although in many cases a stiffness closer to the range of between 100 MPa and 1.2 GPa (or any subrange thereof) is more desirable. For example, in some embodiments a column has a stiffness of between 100 MPa and 2 GPa, 200 MPa and 600 MPa, 200 MPa and 1.5 GPa, 300 MPa and 1.2 GPa, 500 MPa and 850 MPa, or any other subrange between 10 MPa and 100 GPa.

It should be noted that, in accordance with some embodiments, a stiffness of a column 32 during expansion or deployment can include a tradeoff between expansion height, desired force exerted on the implant 30 to obtain the desired distraction (in some cases, approximately 400 N total), and the maximum force to be exerted on the endplate 24 per unit area (in some cases, 4-8 MPa). Thus, in some embodiments, as the implant grows in footprint, the desired expansion stiffness (per unit area) can decrease.

In some embodiments, the implant 30 is configured to have a different stiffness while the implant is being deployed (e.g., while it is transitioning from an insertion configuration to a deployed configuration) than when it is in the deployed configuration. In some cases, the stiffness of the implant while it is being deployed is between 5% and 250% less than the stiffness of the implant in the deployed configuration (or any subrange thereof, such as between 10% and 200% less, 25% and 150% less, 30% and 100% less, 40% and 60% less, or any other subrange). In accordance with some embodiments, the stiffness of the implant when it is being deployed is below or near the fracture pressure of bone, while the stiffness of the implant when fixed is (in some embodiments) below 1.2 GPa.

As discussed in additional detail below, some embodiments include multiple different types of columns 32, such as an expanding column 32a and a fixed column 32b. In some cases, a first column (which can be an expanding column, a fixed column, or any other suitable type of column) has a first stiffness (e.g., any of the stiffnesses discussed above), and a second column (which can also be an expanding column, a fixed column, or any other suitable type of column) has a second stiffness (e.g., any of the stiffnesses discussed above, which can be the same or different from the first stiffness). In some embodiments, a combination of the first stiffness with the second stiffness results in a particular desired stiffness provided by the implant as a whole (e.g., any of the stiffnesses set forth above). By way of non-limiting illustration, in some embodiments, each of the first stiffness and the second stiffness is between 100 MPa and 1.2 GPa (or any subrange thereof). As another example, some embodiments of the first stiffness and the second stiffness combine to equal a combined stiffness of between 100 MPa and 1.2 GPa (or any subrange thereof). Some embodiments include additional columns with the same or different stiffnesses (or combinations of stiffnesses). In some embodiments, the expanding column is configured to have a stiffness that is tailored to its contact area so that the resultant contact pressure is less than the fracture pressure of the bone. In this regard, the exact values can be a design choice and can be enabled by this described geometry.

In some embodiments, the stiffness of the first column 32 (e.g., an expanding column 32a) is different from the stiffness of the second column (e.g., a fixed column 32b). For example, in some embodiments, the first stiffness is less than the second stiffness. That said, even though in some embodiments it is generally advantageous for the first stiffness to be less than the second stiffness, the first stiffness of some embodiments is more than the second stiffness. In some embodiments, the first stiffness is up to 99.9 GPa less (or more, in some embodiments) than the second stiffness. For example, in some cases the first stiffness is up to approximately 90 GPa, 75 GPa, 50 GPa, 25 GPa, 10 GPa, 5 GPa, 3 GPa, 2 GPa, 1 GPa, 900 MPa, 800 MPa, 750 MPa, 700 MPa, 650 MPa, 600 MPa, 550 MPa, 500 MPa, 450 MPa, 400 MPa, 350 MPa, 300 MPa, 250 MPa, 200 MPa, 150 MPa, 100 MPa, 50 MPa, 10 MPa, or any other suitable amount less (or more) than the second stiffness. In some embodiments, the stiffness differential between the first stiffness and the second stiffness falls within a range, such as a range including any two of the foregoing stiffnesses or any other range between 1 MPa and 99.9 GPa. By way of non-limiting illustration, in some embodiments, the second stiffness is between 5 MPa and 1.2 GPa stiffer than the first stiffness (or vice versa). Moreover, in some embodiments, the second stiffness is between 10 MPa and 200 MPa stiffer than the first stiffness (or vice versa).

In some embodiments, by including multiple columns 32, a wide variety of endplate 24 topologies can be accommodated with a single implant 30. Indeed, in some embodiments, the implant causes a desirable load to be exerted across a large percentage of a surface area of an endplate (e.g., more than 50%, 60%, 70%, 80%, 90%, 95%, or 99%, or even up to 100% of the surface area of the endplate) notwithstanding variations in the topology of the endplate or variations between different endplates. In some cases, the desirable load to be exerted across the endplate is between 50 MPa and 5 GPa (or any subrange thereof). For example, in some embodiments, the implant is configured to provide a load of between 300 MPa and 1.2 GPa (±150 MPa) across a large percentage of a variety of different endplates with different topologies (e.g., at least 90%, 95%, 99%, or more of naturally occurring human endplates). Moreover, in some embodiments, the implant is configured to provide a load of between 100 MPa and 4.1 GPa (±100 MPa) across a large percentage of a variety of different endplates with different topologies.

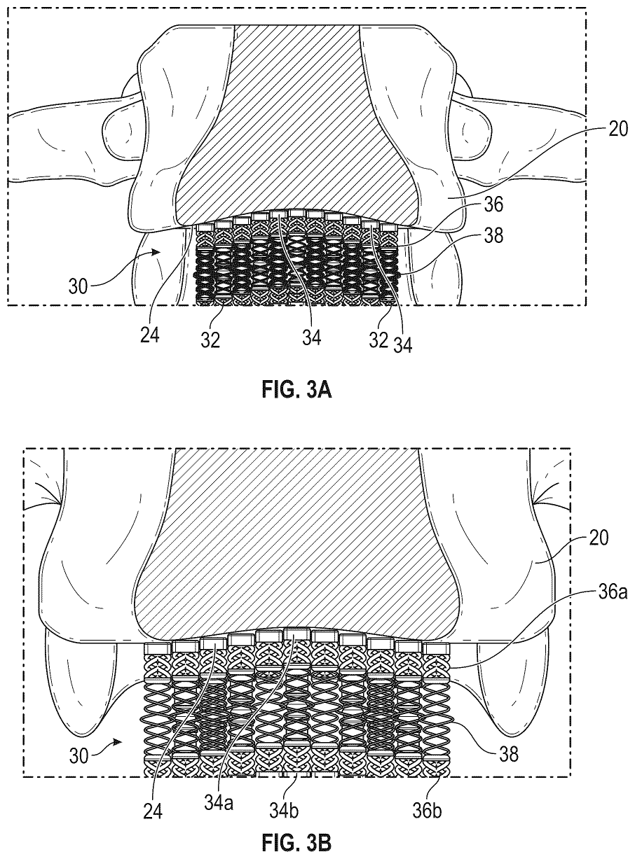

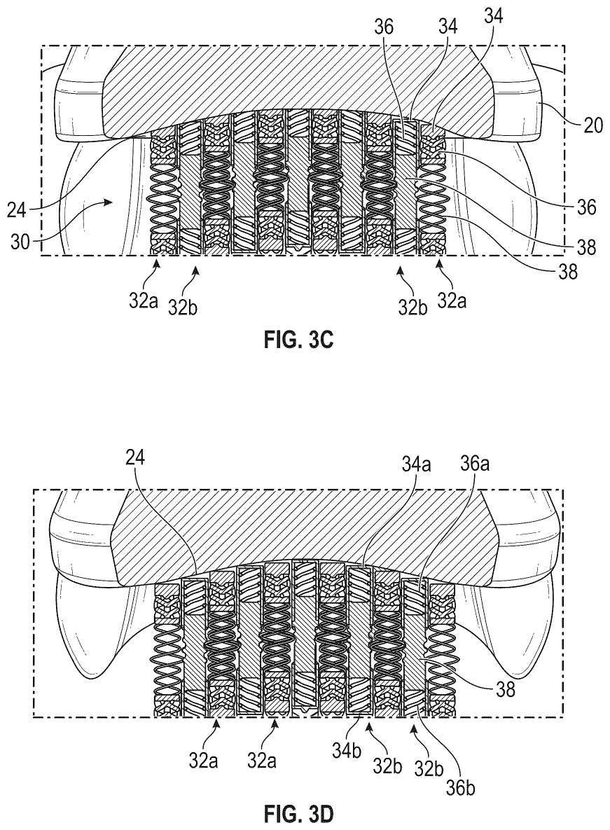

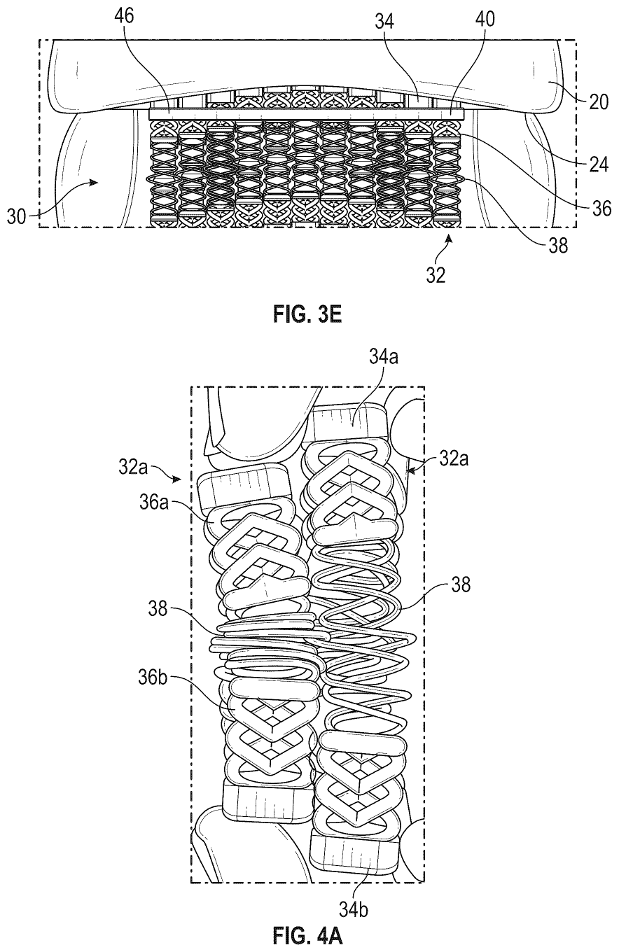

According to some embodiments, the implant 30 is tailored to conform not only to different depths of endplate 24 topology, but also to different regions of the endplate (e.g., the central region, the posterior region, the lateral region, the anterior region, or another region). In this regard, endplates generally vary in stiffness, strength (e.g., load-bearing capacity), and shape across their various regions. In some embodiments, the implant is tailored to meet specific needs depending on these natural variations. For example, in some embodiments, the implant includes one or more regions that correspond with one or more regions of an endplate. In this regard, some embodiments of the implant include one or more surfaces (e.g., a rostral surface, a caudal surface, or any other suitable surface) that include one or more of a central region (e.g., corresponding with the central region of an endplate), a posterior region (e.g., corresponding with the posterior region of an endplate), a left lateral region (e.g., corresponding with the left lateral region of an endplate), a right region (e.g., corresponding with the right lateral region of an endplate), an anterior region (e.g., corresponding with the anterior region of an endplate), or any other suitable region. By way of non-limiting illustration, FIGS. 3A-3E show an expandable and conforming implant 30 with multiple columns 32 conforming to an endplate 24 of a vertebral body 20.

In some embodiments, one or more regions of the implant 30 are modified to have one or more differences, such as a different stiffness, a different size, a different shape, a different contact area, a different pore density, a different material composition, different structure, or another different attribute. By way of non-limiting example, in some embodiments, at least one of a posterior region, a left region, a right region, an anterior region, a central region, or another region of the implant has a greater (or lesser) stiffness than at least one other region of the implant. Where this stiffness is greater, this can allow a greater load to be transferred to the corresponding region of the endplate. In this regard, the central region of the bony endplate can sometimes receive the least amount of load from an implant, as the depth of the topology tends to be greatest at the central region (so the deflection of the corresponding central region of the implant is least, compared to the other regions). Thus, to compensate, some embodiments of the central region of the implant have increased stiffness, increased length (or maximum length), or any other suitable difference, thereby increasing the load applied to the central region of the endplate (and causing a more uniform load to be applied to the endplate overall).

In accordance with the foregoing, some embodiments of the implant 30 have a surface (e.g., formed of multiple ends 34 of columns 32) that is adapted across various portions of the implant. In this regard, differences included in the surface (if any) can be arranged in any suitable manner. In some embodiments, the differences within the surface closely align with the regions of the bony endplate 24. In some embodiments, the surface includes differences within the various regions. For example, some embodiments of the surface include a gradient, with a particular difference (e.g., stiffness) increasing or decreasing medially to laterally, left to right, anterior to posterior, central to exterior, or in another configuration throughout all or a portion of the implant. Some embodiments include differences within the surface arranged in a custom-tailored manner or in another specific arrangement (e.g., in a particular geometric pattern). It is noted that the vertebral endplate is often stiffest at the lateral edges, with decreasing stiffness toward the center and also somewhat toward the ventral aspect. In any case, tailoring the stiffness of the implant to best match the bony endplate can, in some cases, ameliorate risk of bone damage.

In some embodiments, the implant 30 is configured to conform to a single endplate 24, but some embodiments of the implant are configured to conform to multiple endplates or to fill one or more voids in a vertebral body. For example, some embodiments of the implant include multiple surfaces, such as a rostral surface and an opposing caudal surface, a ventral surface and an opposing dorsal surface, or a right surface with an opposing left surface. In this regard, some embodiments of the implant are “double-sided”, with a rostral conforming region on a rostral surface of the implant (configured to conform with a caudal-facing endplate above the implant) and a caudal conforming region on a caudal surface of the implant (configured to conform with a rostral-facing end plate below the implant). As with the various regions (or portions of regions) within a surface (as discussed above), where multiple conforming regions are included, the conforming regions can differ from each other in any suitable manner (including in stiffness, size, shape, contact area, design, pore density, material composition, structure, or any other suitable attributes). In any case, any of the features and characteristics discussed herein can be applied to one or both surfaces or conforming regions.

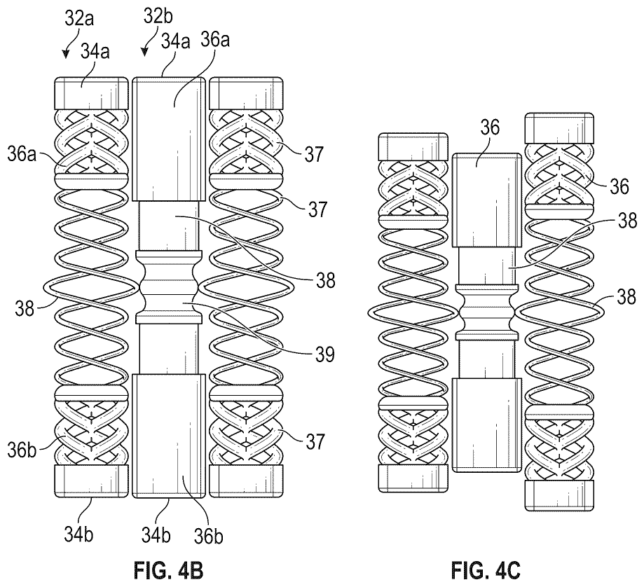

According to some embodiments, the implant 30 is configured to be “sandwiched” between two vertebrae 20. Accordingly, in some cases, when a rostral endplate applies load to an implant endplate (e.g., a rostral surface), the load is applied on the other side as well (e.g., at the caudal surface loaded against a caudal endplate). Yet, the contact pressure between implant and bone can (in accordance with some embodiments) be made to vary across the contact region to match the natural or exceptional variation in bone density.

Where the implant 30 includes multiple columns 32, the columns can include any suitable component and have any suitable configuration that allows them to accomplish their intended purposes. As mentioned above, some embodiments include multiple different types of columns, such as one or more expanding columns 32a, one or more fixed columns 32b, or one or more additional types of columns. In some cases, all columns (including columns of different types) share one or more characteristics, and in some cases, columns of different types have different sets of characteristics. In accordance with the foregoing, descriptions of the various different kinds of columns are provided below, but it is noted that (in accordance with some embodiments) an expanding column can have or lack any characteristic described with respect to a fixed column and vice versa.

Where the implant 30 includes one or more expanding columns 32a, the expanding columns can include any component or have any configuration allowing them to expand or to effectuate an expansion of the implant. In some embodiments, the expanding columns have multiple regions that can differ from each other in stiffness (e.g., in accordance with any of the stiffnesses discussed above), porosity, strength, material, design, or any other characteristics. For example, some embodiments of the expanding columns include one or more outer regions 36 and one or more inner regions 38 (in this case, the term “outer” can mean disposed toward a cranial or caudal surface or end 34 of the column, and the term “inner” can mean disposed at or near a plane positioned halfway between the caudal and cranial surfaces or away from the ends of the column). In some cases, by having different regions of different stiffnesses, the implant is able to have a different effective stiffness while it is being shifted from the insertion configuration to the deployed configuration than when it is in the deployed configuration for everyday use. By way of non-limiting illustration, FIG. 4A shows expanding columns 32a, each having a first outer region 36a disposed proximate to a first end 34a and a second outer region 36b disposed proximate to a second end 34b, with an inner region 38 disposed between the first outer region and the second outer region.

Similarly, Where the implant 30 includes one or more fixed columns 32b, the fixed columns can include any component or have any configuration allowing them to provide support for the implant (e.g., for the expanding columns 32a). In some embodiments, the fixed columns have multiple regions that can differ from each other in stiffness (e.g., in accordance with any of the stiffnesses discussed above), porosity, strength, material, design, or any other characteristics. As with the expanding columns, some embodiments of the fixed columns include one or more outer regions 36 and one or more inner regions 38. By way of non-limiting illustration, FIGS. 4B and 4C show fixed columns 32b alongside expanding columns 32a, each having a first outer region 36a disposed proximate to a first end 34a and a second outer region 36b disposed proximate to a second end 34b, with an inner region 38 disposed between the first outer region and the second outer region.

Although some embodiments of the implant 30 comprise only a single layer of columns 32 (e.g., as shown in FIGS. 4A-4C), in some embodiments, two or more columns are stacked (e.g., arranged in series) in the implant. In such embodiments, any suitable number of columns can be stacked end to end, including 2, 3, 4, 5, 6, or more. Although in some embodiments in which two or more columns are stacked end to end, the columns are not physically locked together, in some other embodiments, two or more columns are physically coupled together, end to end. In such embodiments, the columns can be coupled together in any suitable manner, including through the use of one or more frictional couplings, set screws, mechanical couplings, fasteners, magnets, adhesives, catches, couplers, welds, claps, integrally formed couplings, or in any other suitable manner.

In some embodiments, an end 34 of a column 32 (e.g., a first end 34a or a second end 34b of an expanding column 32a or a fixed column 32b) is configured to contact a bony endplate 24. Accordingly, the end of the column can be configured in any suitable manner to place a load on the endplate, support the endplate, stimulate bone growth, or otherwise positively affect the interaction between the implant 30 and the endplate. For example, the end can have any suitable shape, including circular, semi-circular, triangular, square, rectangular, trapezoidal, pentagonal, hexagonal, star-shaped, T-shaped, polygonal, symmetrical, asymmetrical, or any other regular or irregular shape. By way of non-limiting illustration, FIG. 2B shows columns with various ends 34, including fixed columns 32b having circular ends 34 and expanding columns 32a having square ends 34 with radiused or rounded corners.

In some embodiments, one or more of the ends 34 include one or more surface features 35 for stimulating bone growth or otherwise impacting the ends' interaction with the endplate 24. The surface features can include any suitable surface features, including one or more pores, ridges, grooves, beads, bumps, divots, microstructures, textures, stimulants, hormones, or other features for stimulating bone growth. By way of non-limiting illustration, FIG. 2B shows ends 34 with surface features 35 in the form of pores (which can have a porosity, as described in additional detail in a later portion of this disclosure). In some embodiments, any suitable number of the ends are optionally configured to pierce or extend into the endplate to improve fixation.

In embodiments with columns 32 that include one or more outer regions 36, the outer regions can have any suitable component or configuration that allows them to provide adequate implant support. For example, in some embodiments, the outer region is substantially solid, rigid, and otherwise structurally configured to support the load placed on it by the endplate 24. In some embodiments, the outer region includes a porous structure 37 (as discussed in more detail in a later portion of this disclosure).

In embodiments with columns 32 that include one or more inner regions 38, the inner regions can have any suitable component or configuration that allows them to provide adequate implant support or, in the case of expanding columns 32a, to expand to increase the height of the column or to contract to place the implant 30 in the insertion configuration. For example, in some embodiments, the inner region is substantially solid and rigid (e.g., in the case of fixed columns 32b), and otherwise structurally configured to support the load placed on it by the endplate 24. In some embodiments, the inner region includes one or more porous structures 37 (as discussed in more detail in a later portion of this disclosure). Where the outer region 36 and the inner region 38 each include one or more porous structures 37, the porous structure of the inner region 38 can have different characteristics than the porous structure of the outer region (e.g., a different stiffness, a different porosity, a different strand layout, or another difference). Similarly, the inner region can differ from the outer region in any other respect. For example, some embodiments of the inner region have a diameter that is smaller (or larger) than a diameter of the outer region. Some embodiments of the inner region have a different shape, different contours, a different stiffness, different design, or any other suitable different characteristics. In some embodiments, the inner region includes one or more recesses 39 (which, in some cases, are configured to accommodate one or more strands of a porous region of an adjacent column). In some embodiments, one or more of the fixed columns are configured with, or define one or more pores, or include one or more features to decrease their stiffness and to increase vascularization.

By way of non-limiting illustration, FIGS. 4B and 4C show some embodiments of expanding columns 32a and fixed columns 32b, where the fixed columns have outer regions 36 and inner regions 38 that are each substantially solid and configured to support the load placed on the implant by the endplates. In these illustrated embodiments, the inner region 38 has a smaller diameter than the outer region 36, and it is further contoured, having a recess 39 which can accommodate a strand of a porous structure 37 of a neighboring column. The expanding columns 32a, as illustrated, also have outer regions 36 and inner regions 38, but each of these regions includes a porous structure 37 (the porous structure having thicker strands, and thereby a greater stiffness, at the outer regions). Accordingly, some embodiments of the expanding columns 32a are configured to expand (e.g., from a position in which they have a height approximately equal to a height of the fixed column 32b, as shown in FIG. 4B, to a position in which they have a height greater than the height of the fixed column 32b, as shown in FIG. 4C).

Although the columns 32 and their regions can have any suitable dimensions, in some cases a column (e.g., a fixed column 32b) has a height of approximately 10 mm±8 mm (or any subrange between 2 mm and 18 mm). In some embodiments, an outer region 36 (e.g., a first outer region 36a or a second outer region 36b) of a column (whether a fixed column or an expanding column 32a) has a height of approximately 4 mm±3 mm (or any subrange thereof).

According to some embodiments, the height of one or more columns 32 (including fixed columns 32b, or a deployed or pre-deployed height of expanding columns 32a) is selectively adjustable. In this regard, a column can be configured to be selectively adjustable in any suitable manner, such as by using one or more expandable components, flexible components, telescoping components, threaded components, or any other suitable height-adjustable components of any kind. For example, in some embodiments, the outer regions 36 of a column 32 can include a threaded connection with the inner region 38, such that twisting an outer region with respect to the inner region causes the outer region to move up or down with respect to the inner region, thereby lengthening or shortening the column. When such a system is used in connection with a fixed column, this can (in some embodiments) be useful for adjusting the height of the implant in the insertion configuration. In some cases, the fixed columns have a fixed height, and fixed columns of the desirable height are preselected prior to formation of the implant. Moreover, in some embodiments, a fixed column is expandable (or can be lengthened) such that an expansion of the fixed column is configured to expand less than an expansion of an expanding column.

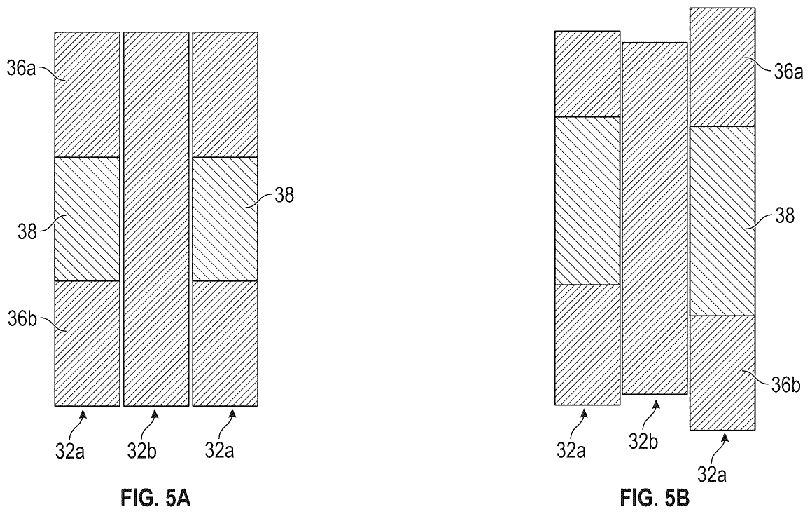

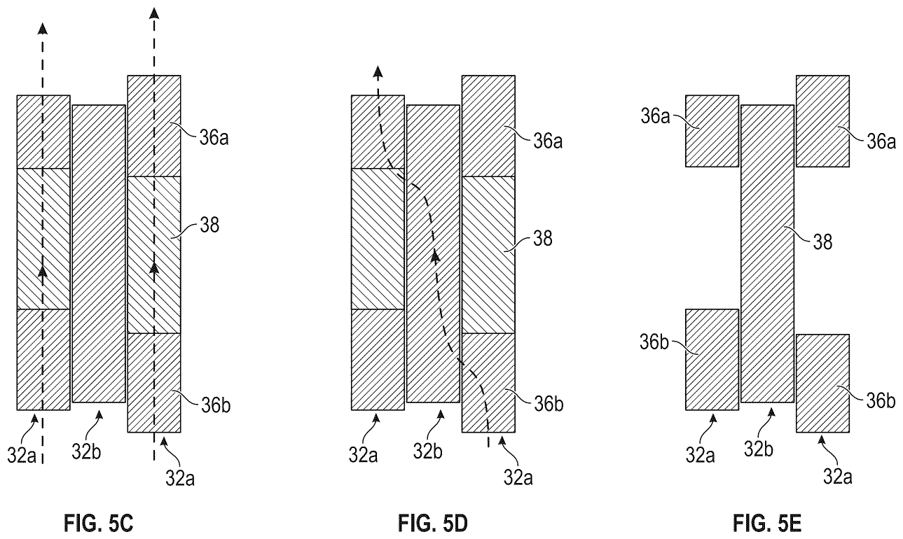

Although deployment of the implant 30 can be accomplished in any suitable manner that causes the overall height of the implant to increase and one or more surfaces (e.g., the caudal surface and the cranial surface) to conform to the respective endplates, according to some embodiments, when the implant 30 is toggled from an insertion configuration to a deployed configuration, the height of the expanding columns 32a increases, while the height of the fixed columns 32b remains the same (e.g., at a pre-insertion height, which, as discussed above, can be preadjusted in some embodiments). For example, FIG. 5A shows an embodiment of a fixed column 32b surrounded by expanding columns 32a, as may be found in an implant in an insertion configuration. FIG. 5B shows an embodiment of the same columns, only in a deployed configuration, in which the inner region 38 of the expanding columns has been allowed to expand, thereby increasing the height of the expanding columns and the overall height of the implant, while the fixed column remains at a constant height.

Where the implant 30 includes one or more expanding columns 32a, the expanding columns can be configured to expand any suitable amount. For example, in some cases, an expanding column is configured to expand from a height of 10 mm (±8 mm) in an unexpanded state to a height of 18 mm (±10 mm) in an expanded state (including any subranges that fall within the foregoing ranges).

According to some embodiments, when the expanding columns 32a are allowed (or caused) to expand, they press against the bony endplates 24 of the vertebral bodies 20 on either side. Accordingly, the load on the endplates is (at least initially) borne by the expanding columns. In some embodiments, the expanding columns are configured to apply a proper load to the implants when they transition from the insertion configuration to the deployed configuration. As discussed above, in some cases, it is desirable for the load to be less during expansion than during everyday loading. Accordingly, some embodiments are configured to have a lesser stiffness during expansion. Thus, generally speaking, if the implant is configured to have a particular stiffness during expansion, the stiffness of any particular expanding column can be such that the stiffnesses of all the expanding columns taken together result in the desired implant stiffness.

In accordance with the foregoing, some embodiments of deploying the implant 30 include expanding the expanding columns 32a (or allowing them to expand) until the expanding columns press against the opposing bony endplates 24 with a desired force (e.g., a desired deployed force—in particular, a force that avoids hyperloading or hypoloading the bony endplates—is achieved). In some embodiments, this is done by manually expanding the expanding columns (e.g., through inflation of a balloon, or otherwise, as described in more detail below). In some embodiments, this is done automatically (e.g., a stiffness of the expanding column, or a stiffness of particular structure thereof (such as the inner region 38), is configured to apply the desired amount of force. For example, if the expanding column includes a spring-like element (e.g., a strand, as discussed below), the spring-like element can be configured to expand until the desired load force is reached. By way of non-limiting illustration, FIG. 5C shows a load (represented by the dashed arrowed lines) being directed through the expanding columns 32a bordering a fixed column 32b.

According to some embodiments, the implant 30 is configured such that the load can be selectively rerouted, transmitted, or otherwise transferred to pass through one or more fixed columns 32b instead of passing entirely through expanding columns 32a after it is deployed. While this can be accomplished in any suitable manner, in some embodiments this is done by coupling the outer regions 36 of the expanding columns to the fixed columns. With the outer regions of the expanding columns coupled to the fixed columns, the load path can pass through the outer regions of the expanding columns and through the fixed columns without needing to pass through the inner regions of the expanding columns (thereby removing the need to continue to rely on the expanding components of the inner regions after the expansion has been effectuated). By way of non-limiting illustration, FIG. 5D shows a pair of expanding columns 32a on either side of a fixed column 32b, with a load path (illustrated by the dashed arrowed line) traveling through a first outer region 36a of a first expanding column 32a a fixed column 32b and a second outer region 36b of a second expanding column 32a.

According to some embodiments, after the outer portions 36 of the expanding columns 32a are coupled to the fixed columns 32b, the inner portions 38 of the expanding columns can be removed. In some cases, the inner portions remain in place, but the load is largely or wholly rerouted away from the inner portions. Accordingly, the stiffness of the implant 30 is transitioned from the stiffness of the expanding columns to the stiffness of the fixed columns, thus allowing for a different stiffness in the deployed configuration than while the implant is being deployed. Furthermore, in some cases, removing the inner portions or rerouting the load can remove variation that could otherwise be caused by the inner portions. For example, certain types of expanding components (e.g., balloons, springs) are very useful for expansion, but they may not be as stable as other components (e.g., having a lesser stiffness can potentially fail to provide the necessary support). That said, some embodiments are configured such that the expanding components continue to bear all or a substantial portion of the load throughout the life of the implant, so rerouting the load is not necessary in every embodiment.

In some embodiments, the inner portions 38 of the expanding columns (e.g., the expanding components) are selectively inserted only as necessary to deploy the implant 30. For example, in some embodiments, the expanding component includes an inflatable element (e.g., a balloon). An example use of such an element is as follows: the expanding element is inserted into the implant after the implant has been inserted into an intervertebral space, and then the expanding component is used to deploy the implant by causing the expanding columns' height to increase, and then the expanding columns are coupled to the fixed columns, and then the expanding element is removed. By way of non-limiting illustration, FIG. 5E shows a fixed column 32b with outer regions 36 of expanding columns 32a fixed thereto, thereby remaining in the expanded configuration notwithstanding the absence of inner regions (not pictured) of the expanding columns.

To summarize some of the foregoing concepts, FIGS. 5A-5E show an example how a deployment of the implant might work: first, the implant is in an insertion configuration, and the expanding columns 32a are unexpanded (e.g., they are the same height as the fixed columns 32b, as shown in FIG. 5A); next, the expanding columns are expanded (e.g., through expansion of the inner regions 38, as shown in FIG. 5B), thereby taking on the load from the endplates (as shown in FIG. 5C) and distracting the disc space; next, the outer regions 36 of the expanding columns are coupled to the fixed columns, such that the load is at least partially rerouted through the fixed columns (as shown in FIG. 5D), effectively strengthening and stiffening the implant to the desired deployed configuration stiffness; next, the inner regions of the expanding columns are removed (or made obsolete), such that the expanded implant bears the load through the fixed columns and the outer regions of the expanding columns (as shown in FIG. 5E). Again, this is just an illustrative example, and variations are possible. For example, in some embodiments, the inner region of the expanding columns continues to bear all or part (e.g., 90%, 80%, 75%, 50%, 25%, 10%, or any other portion) of the load throughout the useful life of the implant.

Where the implant 30 includes one or more expanding columns 32a, the expanding columns can be configured to expand in any suitable manner. Some embodiments utilize an inflatable element, as discussed above (e.g., a specialized balloon configured to expand due to the pressure of a gas blown or pumped into the inflatable element). Some embodiments utilize a mechanical tool (e.g., to pry or otherwise force the first outer region 36a away from the second outer region 36b, thereby increasing the height of the expanding column). In some embodiments, the expanding element is integral to the structure of the expanding column. For example, in some embodiments, a spring-like element (or another element configured to expand under its own power in-situ) is used, which can be compressed to put the implant into the insertion configuration, and which naturally decompresses when allowed to due to the compression on the spring-like element.

In some embodiments that utilize a spring-like element, the spring-like element is configured to remain in an unexpanded (compressed) state until the implant 30 is deployed. While the implant can have any suitable configuration for preventing the expanding column 32a from expanding until the proper time, some embodiments of the expanding column are configured to selectively couple to one or more fixed columns 32b while in the unexpanded state. Accordingly, in some embodiments, the expanding column is configured to automatically expand when decoupled from the fixed column. In some embodiments, the same mechanism used to couple the expanding column to the fixed column when the expanded column is in the expanded state is used to couple the expanding column to the fixed column when the expanded column is in the unexpanded state. That said, in some embodiments, different coupling mechanisms are used for coupling in the expanded state than are used for coupling in the unexpanded state.

Where the expanding column 32a is configured to couple to the fixed column 32b (whether in the expanded state, in the unexpanded state, or both), the coupling can be effectuated in any suitable manner, but some embodiments use one or more couplers 40. Where a coupler is used, the coupler can include any suitable coupling device, such as one or more set screws, eyelets, magnets, hook-and-loop fasteners, adhesives, welds, interference fits, friction fits, tongue-and-groove connections, snaps, ties, pins, rods, bands, rivets, zip ties, or any other coupling mechanisms. In some embodiments, one or more couplers are used to couple other columns 32 together (e.g., a first fixed column to a second fixed column, or a first expanding column to a second expanding column).

In some embodiments with a coupler 40, the coupler includes one or more dovetails 42. Where a dovetail is used, the dovetail can include any suitable tongue-and-groove-type components formed on adjacent columns. For example, in some cases, a first column include a protrusion, and a second column includes a receptacle configured to receive the protrusion. In some embodiments, the dovetail is configured to allow for only a single degree of motion (e.g., in a vertical (caudal-cranial) direction.

In some embodiments with a coupler 40, the coupler includes one or more pins 44. As with the dovetail 42, the pin can include any component configured to restrain movement of adjacent columns 32 with respect to each other. In some cases, the pin is configured to form a friction or interference fit between columns. In some cases, the pin is configured to form a friction or interference fit utilizing a column's structure (e.g., by fitting into a porous structure 37 or a recess 39 of a column). Some embodiments of the pin are configured to restrain movement that would otherwise be allowed (for example, in some embodiments where a dovetail allows cranial-caudal motion, the pin restricts cranial-caudal motion-accordingly, the pin and the dovetail work together to prevent movement of the columns with respect to each other). According to some embodiments, the pin is configured to be selectively removed (and replaced, in some cases) to allow for easy decoupling and recoupling of adjacent columns to each other. Although the pin can have any suitable shape, in some embodiments, the pin is wedge shaped and can be inserted in any suitable direction in order to constrain the column, such as in a medial-lateral, anterior-posterior, or cranial-caudal direction. In some embodiments, the pin (or wedge) is configured to extend into the endplate, thus providing an additional method of securing the implant 30 to the bone.

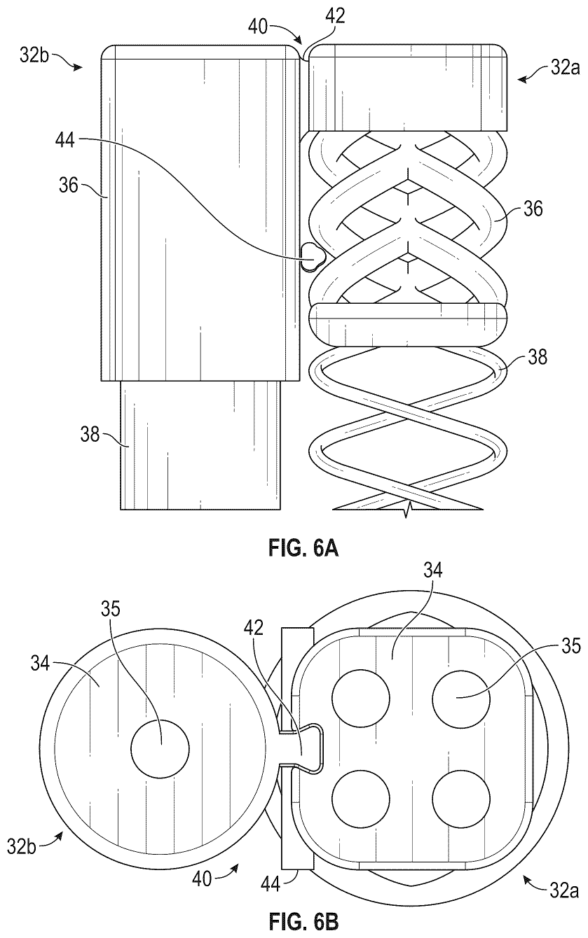

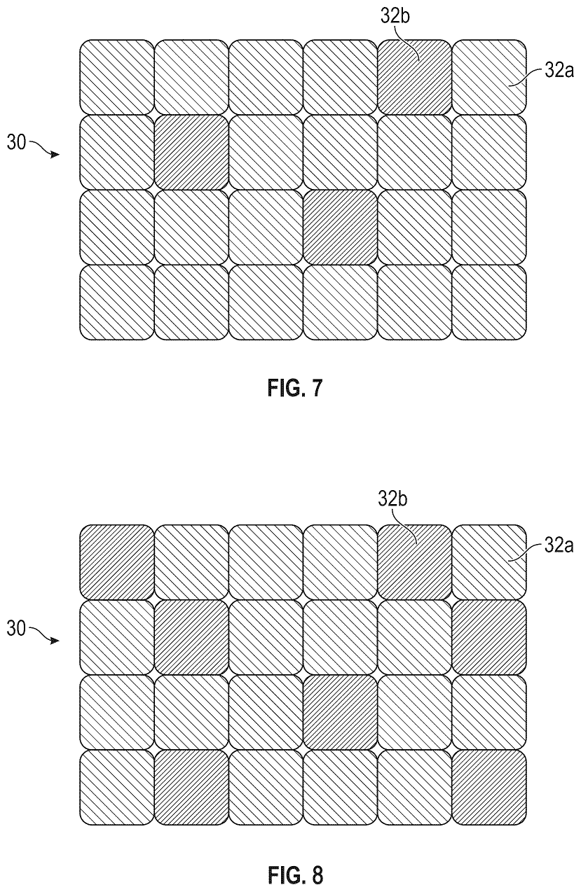

By way of non-limiting illustration, FIGS. 6A and 6B show an expanding column 32a coupled to a fixed column 32b via a coupler 40, where the coupler includes a dovetail 42 in the form of a protrusion and complementary receptacle allowing for movement only in a caudal-cranial direction, as well as a pin configured to interface with the outer region 36 of the expanding column and further configured to restrain movement in the caudal-cranial direction. Accordingly, when the pin is removed, the expanding column will be free to expand (which it will naturally do due to the compression of the spring-like structure of the inner region 38), thereby pushing the outer region cranially (upward).

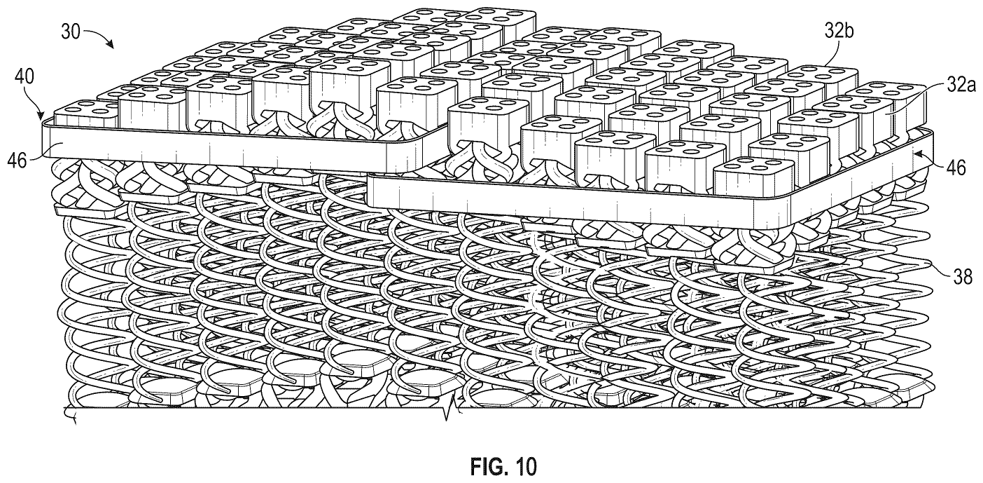

In some embodiments having the coupler 40, the coupler includes one or more bands 46. The band can include any component configured to hold one column 32 to another column (e.g., an expanding column 32a to another expanding column, to a fixed column 32b, or to any number of additional columns). In some embodiments, the band is configured to hold two or more columns together via friction (e.g., by forcing the columns together through a contracting force). The band can be made of any suitable material (e.g., elastic, rubber, metal (with a tight fit), or any other material configured to hold multiple components together by surrounding them (completely or partially) and pushing them together). By way of non-limiting illustration, FIG. 8A shows an implant 30 having a plurality of columns 32a, 32b coupled together by a coupler 40 that includes a band 46. Similarly, FIG. 10 shows an implant 30 with columns 32a, 32b coupled together via a coupler 40 that includes multiple bands 46. Any practical number of bands may be used.

In some embodiments that include a band 46, the band is configured to be selectively loosened and tightened. Accordingly, the band can be loosened to deploy the implant from the insertion configuration to the deployed configuration, allowing all expanding columns 32a to expand, and then the band can be tightened again to ensure that the columns are firmly coupled together (and allowing the fixed columns 32b to take at least part of the load transferred from the outer regions 36 of the expanding columns).

In some embodiments, the coupling is effectuated via one or more phase changes. For example, in some cases, the coupling is accomplished via a polymer melting under an inductive load, Nitinol or another compound undergoing a phase change, application of an adhesive, or another phase-change-related coupling.

According to some embodiments, a retention force (e.g., due to partial application of a coupler 40 or otherwise) is used to limit or slow the expansion of the implant 30. In some embodiments, the retention force is applied in localized regions to encourage formation of curvature (e.g., in the spine, such as lordosis, kyphosis, or other curvature).

Where the implant 30 is configured to be deployed (e.g., toggled from the insertion configuration to the deployed configuration), the deployment can be activated in any suitable manner. For example, as discussed above, the deployment of some embodiments is configured to be activated by changing the configuration of a coupler 40 (e.g., loosening a band, removing a pin, twisting a portion of the coupler, decoupling a portion of the coupler, or otherwise changing the coupler's configuration). In some embodiments, deployment is activated in another way, such as by activating an actuator (e.g., via pressing a button, flipping a switch, turning a dial, or otherwise activating the actuator), inflating an inflatable element, or activating an anchor deployment mechanism. In some cases where an anchor deployment mechanism is used, the anchor deployment mechanism can be used to wedge a component between the sundry columns 32 and an outer ring, band 46, or any other suitable coupler to compress-lock (or otherwise couple) the columns to each other. In some such cases, actuating the anchor deployment mechanism actuates the expansion features. Thus, an anchor deployment mechanism is, in some cases, employed as a wedge between the band and the columns to selectively loosen and tighten (e.g., to effectuate and control deployment). Examples of suitable anchor deployment mechanisms are included in a later portion of this disclosure.

Where the implant 30 includes expanding columns 32a and fixed columns 32b, the columns can be arranged in any suitable manner. For example, the columns can be dispersed evenly, randomly, in a geometric pattern, in an optimized fashion as determined by patient-specific or spinal-segment-specific analysis, or in any other manner. In some embodiments, each expanding column is positioned adjacent to at least one fixed column. In some embodiments, each fixed column is positioned adjacent to at least one expanding column. In some embodiments, each expanding column is positioned adjacent to at least one other expanding column. In some embodiments, the columns alternate (like squares on a chess board), with each expanding column being positioned adjacent to only fixed columns, and vice versa. In some embodiments, all columns on an external perimeter of the implant are expanding columns. In some embodiments, all central columns are expanding columns. In some embodiments, the arrangement of expanding columns and fixed columns corresponds to (or complements) the natural endplate 24 organization. In some embodiments, each expanding column is adjacent to at least one fixed column or at least one expanding column that is itself adjacent to at least one fixed column. Further, although some embodiments are arranged generally in a square grid, other possible arrangements include a hexagonal grid, another type of tessellating or non-tessellating grid, with no specific grid, with a variable grid (e.g., with columns offset from each other instead of aligned edge-to-edge), in an arrangement that is tailored to one or more particular bones, in randomized arrangement, or in any other suitable manner. In short, many different arrangements are possible. By way of non-limiting illustration, FIGS. 7 and 8 show example dispersions of expanding columns 32a and fixed columns 32b within an implant 30.

Some embodiments of the implant 30 (e.g., any column 32 of the implant or any part thereof) can have any suitable characteristic that allows the implant to function as intended, including being planar, contoured, defining one or more openings, being porous, or having any other suitable characteristics. Indeed, some embodiments of the implant include one or more porous structures 37. In this regard, a properly configured porous structure can be highly beneficial in promoting healthy bone growth. Indeed, the cells generally responsible for bone growth, namely osteocytes and osteoblasts, typically work together to form bone as needed within the body. But these cells will typically only form bone under proper conditions, including when the cells experience proper loads and stresses, when a network of blood vessels is available to supply needed nutrients, and when gaps to be filled by bone are of a proper size. When proper conditions are not available, bone often cannot or will not grow. For example, when bone does not experience proper loading (e.g., due to hypoloading), it generally will not grow and can even be resorbed. Additionally, when gaps (i.e., pores) to be filled are too large, bone may not be able to bridge the gaps and often will not grow. Similarly, if pores are too small, bone growth may be impeded by the insufficient size. Thus, some embodiments of the implant employ a combination of a porous structure with proper loading (e.g., as a result of the conforming region) to stimulate improved bone growth.

Where embodiments include a porous structure 37, the porous structure can include any structure having gaps, spaces, air pockets, voids, cavities, hollows, or other pores capable of promoting bone growth (e.g., cortical bone growth, trabecular bone growth, or other bone growth).

According to some embodiments, the porous structure 37 includes one or more pluralities of pores. The pores can include any gaps, spaces, voids, air pockets, cavities, hollows, or other pores that are defined by the porous structure. For example, a hole in a strand qualifies as a pore in some embodiments, and a gap between multiple strands (or multiple portions of the same strand) qualifies as a pore in some embodiments.

In some embodiments, each pore of the plurality of pores has a pore size, or the plurality of pores has an average pore size, of between 10 μm and 3,000 μm or within any subrange thereof. For example, in some embodiments, the plurality of pores each have a pore size, or they have an average pore size, of between 100 μm and 1,000 μm, between 150 μm and 700 μm, between 150 μm and 650 μm, or any other subrange of between 10 μm and 3,000 μm. That said, a pore size of between approximately 150 μm and 650 μm (±100 μm) may be particularly useful for promoting bone growth in combination with the loads placed on bony endplates 24 as a result of the stiffnesses selected for the columns 32 of some embodiments of the implant 30 (as discussed above). In accordance with some embodiments, pores larger than 450 μm can allow for vascularization and pores smaller than 650 μm can allow for bone bridging. Additionally, in some embodiments, areas or pores larger than 650 μm can be selectively placed to increase radiolucency.

The plurality of pores can include any number of pores (e.g., between 2 and 50,000, or any subrange thereof, such as 100±50, 1000±500, 1500±750, 10,000±5000, or any other desired number of pores for promoting bone growth). The pores can also be positioned in any manner helpful for promoting bone growth, but in some embodiments, they are exposed at one or more surfaces of the implant 30 (e.g., any or all of the caudal surface, the cranial surface, the ventral surface, the dorsal surface, the left surface, or the right surface), which in some cases allows bone to access (e.g., grow into) the pores or allows for fluid to circulate within the implant.

In some embodiments, the plurality of pores is interconnected (e.g., each of the plurality of pores is fluidically interconnected with each of the other of the plurality of pores). In some embodiments, the implant 30 (or any particular column 32 of the implant) includes multiple porous structures 37 (e.g., multiple pluralities of pores), which in some cases are interconnected with each other, and in other cases are not interconnected with each other (although the pores within a plurality may still be internally interconnected). For example, in some embodiments, one or more columns 32 includes a porous structure, while one or more other columns do not include a porous structure (in some cases, expanding columns 32a include porous structures, but fixed columns 32b do not). By way of non-limiting illustration, FIG. 4B shows a fixed column 32b without a porous structure and expanding columns 34a that each include multiple porous structures 37 (a porous structure at the first outer region 36a, a porous structure at the second outer region 36b, and a porous structure at the inner region 38).

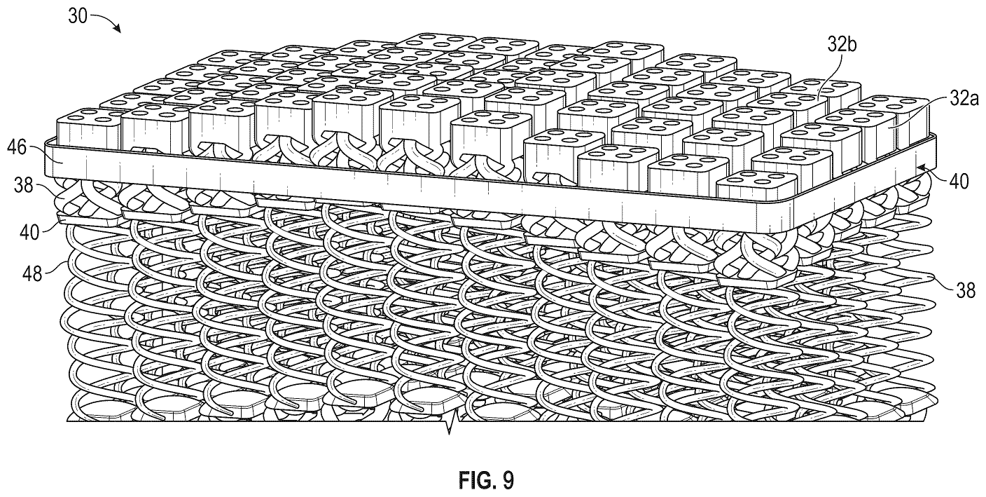

According to some embodiments, the pores of the porous structure 37 are defined by one or more strands 48. The strands can include any component or material capable of defining pores, such as one or more meshes, grids, filaments, foams, lattices, wires, grates, strings, springs, coils, webs, laces, filigrees, frames, frets, screens, reticulations, or other porous components or components that can define pores around them (whether they form strips, porous bodies, or other structures or configurations). By way of non-limiting illustration, FIG. 9 shows an implant 30 having multiple different types of strands 38 (which, in FIG. 9, are generally interwoven, interconnected, filamentous, and spring-like). It may be helpful to note that, generally speaking, each cell in a bone must experience a specific amount of strain for that bone cell to be healthy. In this regard, the spring-like strands included in some embodiments of the implant allow for localized strain on a nano-scale rather than just global strain on a macro-scale (i.e., the stiffness per unit area would be the same for any region of the implant). It should also be noted that some embodiments of the described implant use large voids to allow for a calculated global stiffness per global area that is different in concentrated areas of no voids.

Where the porous structure 37 includes one or more strands 48, the strands can have any configuration suitable for forming the porous structure. For example, in some embodiments, the strands (or portions thereof) are straight, curved, spiraled, twisted, zig-zagged, accordioned, branching, randomly dispersed, geometrically arranged, interwoven, interconnected, separate, independent, uniform, crossed with other strands, woven together, parallel to each other, diagonal to each other, placed with any suitable orientation with respect to each other, varied, or otherwise configured. In some embodiments, one or more of the strands are arranged in a spring-like configuration such that a stiffness is derived from compression of the strands. According to some embodiments, a combination of the material from which the porous structure is formed and the spring-like shape of the material results in a spring constant that yields the desired stiffness of the implant 30 (or the stiffness of one or more of the implant's columns 32 or load bearing regions).

In embodiments in which the porous structure 37 comprises strands 48, the strands can also extend through any portion of the porous structure and in any orientation. For example, the strands (individually or collectively) can extend through the whole porous structure or through only a portion of the porous structure.

Where a strand 48 includes a spring-like configuration, any shape can be used that results in spring-like action of the strand. For example, some embodiments of the strand include a configuration of a torsion spring, a coil spring, a conical spring, a compression spring, a helical spring, a leaf spring, a triangular spring, a rectangular spring, a diamond spring, a spring with another geometric configuration, a single-wire spring, a multi-wire spring, a spring grid, any spring as discussed in the PCT application publication WO2017100366A1 (entitled “Porous interbody spacer”) which is incorporated herein by reference in its entirety, or another type of spring. Furthermore, multiple strands operating as springs can operate dependently of each other (e.g., they are interconnected or coupled in a series configuration, as may be seen with multiple porous structures in a single column 32) or independently of each other (e.g., they are not interconnected, or they are coupled in a parallel configuration, as may be seen with porous structures in different columns). Even where strands are not interconnected (e.g., not physically joined together), strands can be interwoven with each other, such that one strand passes through a negative space not occupied by the other strand (but within the boundaries defined by such strand) without connecting to (or, in some cases, without even contacting) the other strand.

The porous structure 37 can have any number of strands 48, which can be part of one or more groups of strands having similar characteristics. For example, in some embodiments, the first strand is interconnected with additional strands (which, in some cases, have a similar size, shape, stiffness, design, or other configuration as the first strand). Similarly, in some embodiments, the second strand is interconnected with one or more additional strands (which, in some cases, have a similar size, shape, stiffness, design, or other configuration as the second strand). In some cases, maximizing surface area for bone attachment can benefit from having a substantial amount of exposed metal (or any other suitable material used to form the implant 30), which can (in some embodiments) be maximized by keeping the diameter or width of each strand at a minimum to increase the total number of strands. Indeed, some embodiments have between 1 and 10,000 strands, or any subrange thereof. In some cases, a substantial number of strands is generally preferred. For example, some embodiments have between 10 and 5,000 strands, 20 and 500 strands, 50 and 200 strands, or any other number of strands. Indeed, some embodiments (e.g., some lumbar embodiments) comprise about 200 strands±30 strands, while some embodiments (e.g., some cervical embodiments) comprise about 80 strands±30 strands.

In some embodiments, multiple different types of strands are used for different porous structures. For example, in some cases, thicker or stiffer strands are used for segments with a higher stiffness. By way of non-limiting illustration, FIG. 9 shows an implant 30 with a plurality of expanding columns 32a, each having at least one outer region 36 that includes a first strand 48 and an inner region 38 that includes a second strand 48, and the first strand is substantially thicker than the second strand so that the outer region has a substantially higher stiffness than the inner region.

In some embodiments, multiple porous structures 37 (e.g., of adjacent columns 32 or of a single column) are interconnected, but in some embodiments they are separated from one another. For example, some embodiments include one or more dividers 50 to separate porous structures from each other, to separate regions (e.g., an outer region 36 and an inner region 38) of a column 32 from each other, or to separate any other components or structures. Such dividers can have any suitable component for separating or delineating between components (e.g., one or more sheets, rods, barriers, gratings, meshes, lattices, nets, barriers, walls, or other dividing components). By way of non-limiting illustration, FIG. 9 shows an implant 30 with dividers 50 positioned between outer regions 36 and inner regions 38 of expanding columns 32a.

According to some embodiments (and as mentioned earlier), the implant 30 includes one or more anchor deployment mechanisms. In this regard, an anchor deployment mechanism can include any suitable mechanism that is configured to deploy one or more screws, pins, blades, or other anchors configured to fix the implant within the intervertebral space 22 (e.g., by pressing into or otherwise coupling to a bony endplate 24). As mentioned above, the anchor deployment mechanism of some embodiments is functionally coupled to an expansion deployment mechanism of the implant, such that deployment of one or more anchors also effectuates expansion of the expanding columns 32a. Although any suitable anchor deployment mechanism can be used, some embodiments use a mechanism as taught in U.S. Pat. No. 11,944,552 B2, entitled STAND-ALONE INTERBODY FUSION, filed Mar. 8, 2019, which is incorporated herein by reference in its entirety, or as taught in any of the provisional applications to which this disclosure claims priority. In some embodiments, such a mechanism is modified (in any suitable manner) to function without interfering with one or more columns 32, to interact with a coupler 40, or to otherwise operate in connection with the systems and methods described herein.

According to some embodiments, springs (e.g., for use in the expanding column 32a) are selected or formed to have particular stiffnesses or spring constants in accordance with one or more of the following equations:
Equation 1: In a compression spring, deflection is caused by twisting the wire diameter, and therefore the spring constant (k) is as follows:

$$k = \frac{{Gd}^{4}}{8 {N}_{α} {D}^{3}}$$

Equation 2: Torsional stress of compression springs:

$$τ ∘ = \frac{8 DP}{π {d}^{3}}$$

The described systems and methods can be modified in any suitable manner. For example, although some embodiments of the implant 30 are specifically configured to be used in spinal surgery, some embodiments are configured to be used in other parts of the body (e.g., in joint replacements, bone grafts, or other types of surgery). Accordingly, some embodiments of the implant are not limited to being spinal implants. Indeed, in some embodiments, the implant is configured for use with one or more acetabular cups. Additionally, in some cases, the described systems and methods are modified for use with a femoral stem-allowing the expansion to occur in a radial direction.

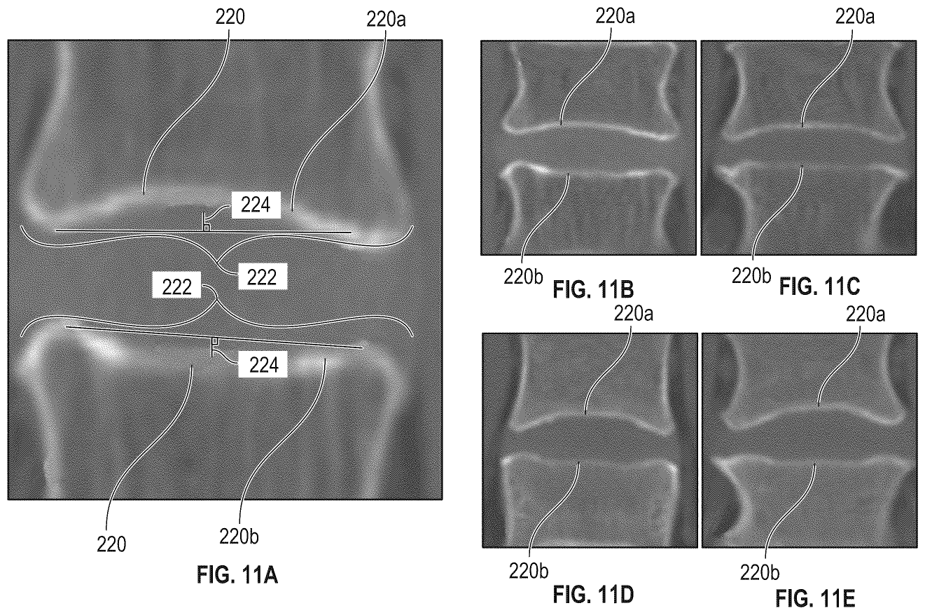

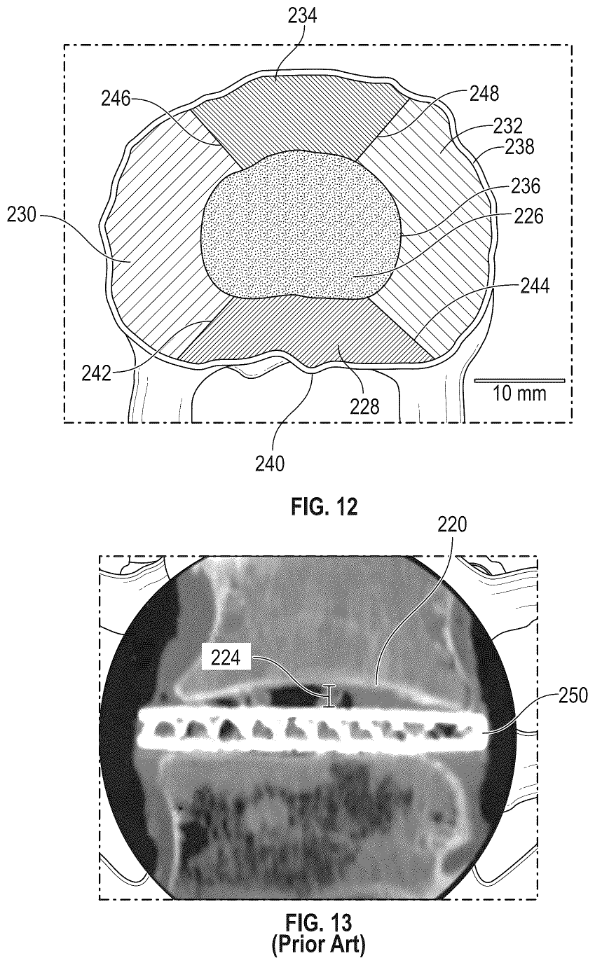

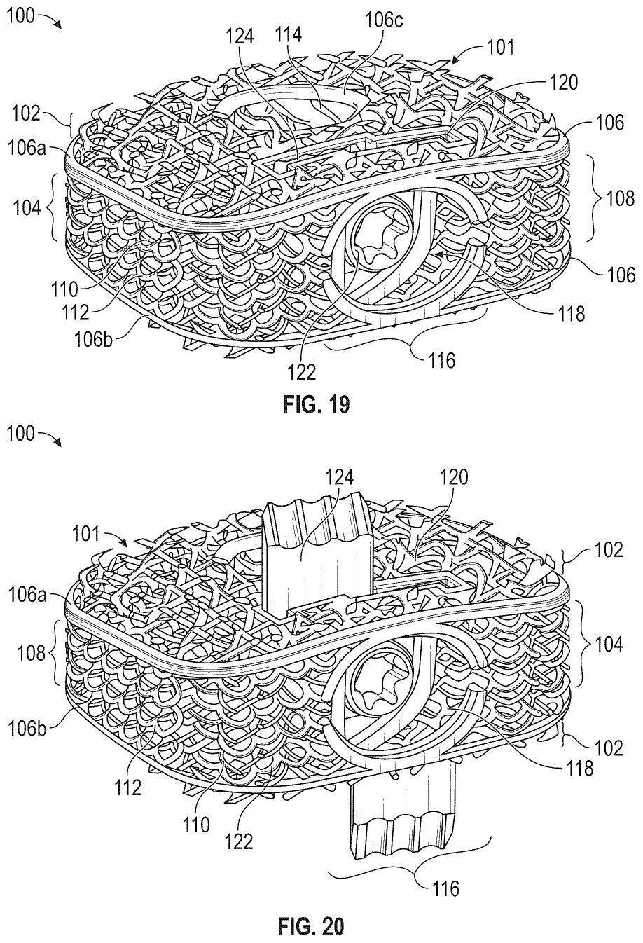

In addition to the aforementioned features, the described systems and methods can include any other suitable feature. For example, some such additional features include one or more supplementary anchors (to anchor the implant 30 in place, such as by affixing the implant to one or more vertebral bodies 20). Other additional features can include protein coatings or other use of biological materials to stimulate bone growth. Other features, which can be used instead of or in addition to any of the features discussed above are further described below (with general reference to FIGS. 11A-21).

Many traditional implants do not conform well to natural structures in a spinal cord, such as bony regions. For example, traditional spinal implants do not always conform well to a patient's natural endplates of the patient's spinal segments. Such conformity failures can result in voids or gaps that are slow to backfill, localized regions of high contact stress that lead to bone loss and subsidence, and other serious issues that can cause loss of function, pain, and decreased quality of life for patients.

To provide some context for natural variation of bony surfaces as discussed herein, FIG. 11A illustrates that bony endplates (endplates) 220 of spinal segments (including upper or rostral endplates 220a and lower or caudal endplates 220b) typically each have their own unique topology 222. Such topology can vary greatly between endplates across patients and even across spinal segments of a single patient. For example, the topology of a bony endplate often forms a bowl shape, a plate shape, a parabolic dish shape, or another curved configuration. Such curvature, however, can vary substantially. In this regard, the topology of a given endplate generally has a depth 224 (or a distance from a most raised portion to a most depressed portion of the endplate), which may range from anywhere between 0.01 mm and 10 mm (or any subrange thereof). For example, in FIG. 11B, the topology of the rostral (upper) endplate 220a has a depth of approximately 3.6 mm, while the topology of the caudal (lower) endplate 220b has a depth of approximately 2.6 mm. In FIG. 11C, the rostral endplate's 220a depth is about 3.4 mm, while the caudal endplate's 220b depth is about 0.4 mm. In FIG. 11D, the rostral endplate's 220a depth is about 5.1 mm, and the caudal endplate's 220b depth is about 2.2 mm. In FIG. 11E, the rostral endplate's 220a depth is approximately 6.3 mm, and the caudal endplate's 220b depth is about 1.4 mm. Other depths and combinations are also possible.

In some cases, in addition to having a particular depth 224, the topology 222 of an endplate 220 fits into a particular topological category, such as shallow-shallow (as illustrated by the rostral endplate 220a in FIG. 11B), shallow-flat (as illustrated by the rostral endplate 220a in FIG. 11C), deep-shallow (as illustrated by the rostral endplate 220a in FIG. 11D), deep-flat (as illustrated by the rostral endplate 220a in FIG. 11E), or other categories. In some cases, the topology does not necessarily fit well into a particular category.

Moreover, the topology 222 of an endplate 220 can also have regional variations. Generally speaking (and as shown in FIG. 12), an endplate can be divided into a central region 226, a posterior region 228, a left region 230, a right region 232, and an anterior region 234. Each region can be defined with respect to its position on the endplate. For instance, a central boundary 236, which separates the central region from the outer regions (the posterior, anterior, left, and right regions), can be located approximately 50% of the way between a center point of the endplate and a perimeter of the endplate (±10%). The outer regions can terminate at an outer boundary 238, which can be located at the perimeter, or, in some cases, spaced apart from the perimeter by a gap (for example, 95% of the way from the center point to the perimeter (±5%)). In some cases, the positions of the outer regions are defined with respect to a particular point, such as a median point 240, located at the posterior region and defined as 0°. With respect to this point, a left posterior boundary 242 (separating the left region from the posterior region) can be positioned approximately 45° clockwise from the median point (±) 10°. A right posterior boundary 244 (separating the right region from the posterior region) can be positioned at approximately 315° (again, +) 10°. A left anterior boundary 246 (separating the left region from the anterior region) can be positioned at approximately 135° (±) 10°. A right anterior boundary 248 (separating the right region from the anterior region) can be positioned at approximately 225° (±) 10° clockwise of the median point. Generally, each region has its own natural variations, thereby leading to a highly diverse possible range of topologies.

In any case, each endplate 220 tends to have its own unique topology 222, with its own particular microscopic and macroscopic variations. Accordingly, many traditional spinal implants-which generally have no ability (or only limited ability) to conform to topological variations-often do not fit well with different endplates. Indeed, as shown in FIG. 13, non-conforming implants 250 tend to create regions of hyperloading, or excessive strain, such as where a portion of an endplate 220 directly contacts the rigid implant. Hyperloading can result in micro or macro fractures in the patient's spinal segments, causing pain and potentially leading to complications requiring future surgery. Similarly, existing implants also tend to create regions of hypoloading, such as where there are gaps between the implant and the body endplate (e.g., due to the endplate's depth 224). Hypoloading can lead to bone atrophy, instability of the implant, or other issues. In short, many traditional implants do not create even loading across spinal endplates having varying topologies. These shortcomings can lead to a patient experiencing large amounts of pain, reduced physical capacity, and other major problems.

Some embodiments of the instant systems and methods address the shortcomings discussed above by providing an implant 100 having one or more conforming regions 101 configured to conform to a variety of topologies 222. In this regard, the conforming region can have any configuration suitable for conforming to a variety of topologies, such as by including one or more springs, strands, sponges, foams, filaments, flexures, lattices, grids, springs, trusses, scaffolds, seals, other strands, layers of the foregoing, or any other suitable components capable of conforming to one or more uneven surfaces. That said, conformity alone is often not sufficient. Rather, an implant often needs to provide a certain amount of support, so a highly conforming component on its own (e.g., a cotton ball) would likely be unable to provide the necessary support in many cases, and thus would be unable to function as a suitable implant in such situations. Accordingly, some embodiments of the instant systems and methods include an implant with one or more conforming regions (e.g., the rostral or caudal face (or endplate) of the implant) as well as a rigid region 103 (e.g., the implant body) that is less conforming than the conforming region.

To illustrate the foregoing, some embodiments of the implant 100 have one or more load-bearing regions (such as a first load-bearing region 102, a second load-bearing region 104, and any number of additional load-bearing regions). In some cases, various load-bearing regions have different stiffnesses, thus giving rise to one or more conforming regions 101 and one or more rigid regions 103.

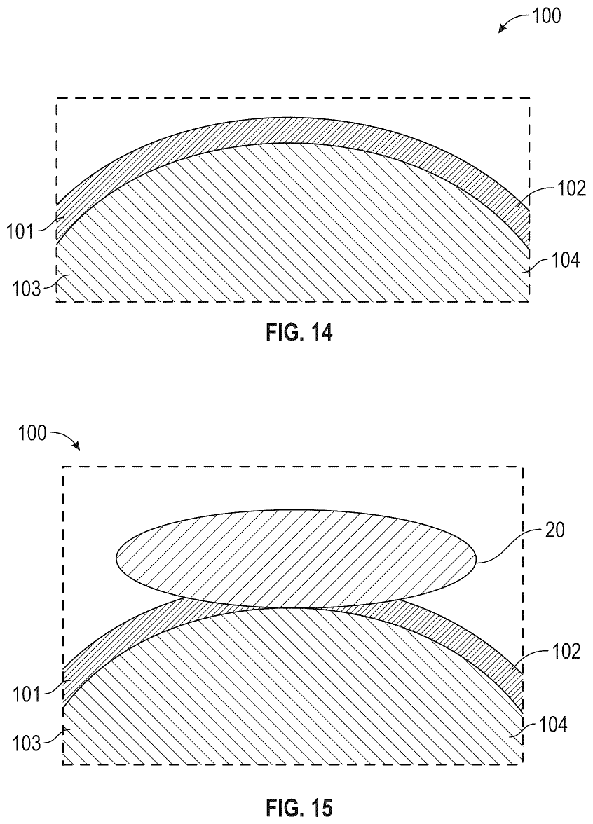

An example of how an implant 100 can accommodate a variety of topologies 222 is shown in FIGS. 14-15, in which an implant 100 has a conforming region 101 that includes a first load-bearing region 102. In the implant 100, the first load-bearing region 102 is provided along with a second load-bearing region 104 that has a greater stiffness than the first load-bearing region 102. Accordingly, as shown in FIG. 15, when a portion of a body endplate 220 comes into (direct or indirect) contact with the implant 100, the conforming region 101 compliantly deforms to accommodate the endplate 220. In this manner, the first load-bearing region 104 provides better support for the endplate's contours (e.g., preventing hyperloading on the points that protrude the farthest), while the second load-bearing region 104 (with a greater stiffness) does not deform as easily, thereby providing adequate support for the endplate (e.g., preventing hypoloading).

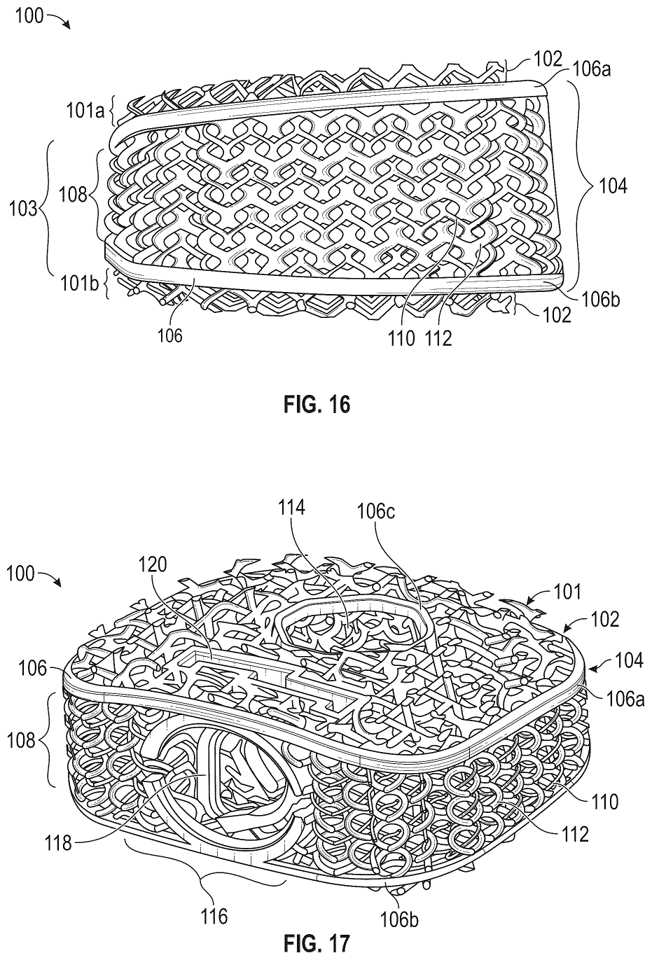

The conforming regions 101 (e.g., the first load-bearing region 102) and the rigid regions 103 (e.g., the second load-bearing region 104) can have any suitable component that allows them to function as described herein. Indeed, in some embodiments (e.g., as shown in FIGS. 16-17), the conforming regions 101 (e.g., the first load-bearing region 102) and the rigid regions 103 (e.g., the second load-bearing region 104) are formed of or otherwise include a variety of implant components, such as one or more frames 106, one or more porous structures 108 (which may include one or more strands, such as a first strand 110, a second strand 112, and any number of additional strands), one or more passages 114, one or more primary fixation mechanisms 116 (which can include one or more: receptacles 118, slots 120, actuation mechanisms 122, or anchors 124), or one or more additional features (e.g., contact features, support features, passages, bodies, faces, cavities, pores, membranes, or other implant features) that may be used in connection with a conforming implant or used to cause the implant to conform (or aid the implant in conforming) to a variety of topologies.

As mentioned above, some embodiments of the described implant 100 include one or more load-bearing regions (e.g., a first load-bearing region 102, a second load-bearing region 104, a third load-bearing region, a fourth load-bearing region, a fifth load-bearing region, and any other suitable number of additional load-bearing regions). The load-bearing regions of the implant can include any suitable component configured to place a load upon any portion of a spinal segment endplate. In this regard, it is often desirable to place a particular load on an endplate to avoid hyperloading or hypoloading (as discussed above) and the associated problems. Thus, the load-bearing regions can have any features or attributes allowing them to (alone or in combination) place the proper loads on the endplates.

The load-bearing regions (e.g., the first 102 and second 104 load-bearing regions) can be formed of or otherwise include any suitable material or materials. For example, some embodiments include one or more of metal (e.g., titanium, steel (e.g., stainless steel or other steel), tantalum, cobalt, chromium, magnesium, platinum, copper, aluminum, or any other biocompatible metal or metal alloy), polymer material (e.g., polyethylene, polyamides, polyurethane, polycarbonate, polypropylene, polystyrene, polyvinyl chloride (PVC), acrylonitrile butadiene styrene (ABS), polylactic acid (PLA), polyetheretherketone (PEEK), or any other biocompatible polymer material), glass, plastic, carbon fiber, wood, fabric, ceramic, fiberglass, or any other material.

Where the implant 100 comprises more than one load-bearing region (e.g., the first 102 and second 104 load-bearing regions), the various load baring regions can all comprise the same, different, or any suitable combination of material or materials. Indeed, in some embodiments, a first load-bearing region 102 is formed of or otherwise includes a first material or combination of materials, and a second load-bearing region 104 is formed of or otherwise includes a second material or combination of materials. In some cases, the first material or combination of materials is the same as the second material or combination of materials, but in some cases it is different. Similarly, a third load-bearing region can include a third material (or combination of materials), which can be the same as or a different material from the first or second materials (or combinations), as can a fourth load-bearing region, a fifth load-bearing region, and any other suitable number of additional load-bearing regions.

The load-bearing regions (e.g., the first 102 and second 104 load-bearing regions) can also include any suitable configuration allowing them to load against (e.g., apply pressure, directly or indirectly, to) a spinal segment endplate. In some embodiments, the configuration of the load-bearing regions (alone or in combination) allows the implant 100 (or one or more portions thereof) to conform to a variety of endplate topologies and to place a satisfactory load on a variety of endplates (without hypoloading or hyperloading the endplate or portions thereof) despite topological variations of such endplates. By way of example, some embodiments of the load-bearing regions include one or more solid components, hollow components, porous regions, foamy regions, frame regions, spring regions, fibrous regions, filamentous regions, foam regions, spongy regions, flexures, grids, seal regions, cushion regions, sheets, columns, curves, geometrical shapes, lattices, scaffolds, or any other suitable configuration of one or more features that are configured to impart particular stiffnesses, strengths, or any other suitable attributes to the load-bearing regions. Some examples of specific configurations of some suitable load bearing regions are provided in a later portion of this description below.

The load-bearing regions of the implant 100 can have any suitable stiffnesses for conforming to an endplate and exerting a suitable load on the endplate. According to some embodiments, one or more of the load-bearing regions is configured to have a stiffness between 10 MPa and 100 GPa (or any subrange thereof), although in many cases a stiffness closer to the range of between 300 MPa and 1.2 GPa (or any subrange thereof) is more desirable. For example, in some embodiments a load bearing region has a stiffness of between: 100 MPa and 2 GPa, 200 MPa and 1.5 GPa, 300 MPa and 1.2 GPa, 500 MPa and 850 MPa, or any other subrange between 10 MPa and 100 GPa.

In some cases, a first load-bearing region 102 has a first stiffness (e.g., any of the stiffnesses discussed above), and a second load-bearing region 104 has a second stiffness (e.g., any of the stiffnesses discussed above, which can be the same or different from the first stiffness). In some embodiments, a combination of the first stiffness with the second stiffness results in a particular desired stiffness provided by the implant as a whole (e.g., any of the stiffnesses set forth above). By way of non-limiting illustration, in some embodiments, each of the first stiffness and the second stiffness is between 300 MPa and 1.2 GPa (or any subrange thereof). As another example, some embodiments of the first stiffness and the second stiffness combine to equal a combined stiffness between 300 MPa and 1.2 GPa (or any subrange thereof). Some embodiments include additional load-bearing regions with the same or different stiffnesses (or combinations of stiffnesses).

In some embodiments, the first stiffness of the first load-bearing region 102 is different from the second stiffness of the second load-bearing region 104. For example, in some embodiments, the first stiffness is less than the second stiffness. That said, even though in some embodiments it is generally advantageous for the first stiffness to be less than the second stiffness, the first stiffness of some embodiments is more than the second stiffness. In some embodiments, the first stiffness is up to 99.9 GPa less (or more, in some embodiments) than the second stiffness. For example, in some cases the first stiffness is up to approximately 90 GPa, 75 GPa, 50 GPa, 25 GPa, 10 GPa, 5 GPa, 3 GPa, 2 GPa, 1 GPa, 900 MPa, 800 MPa, 750 MPa, 700 MPa, 650 MPa, 600 MPa, 550 MPa, 500 MPa, 450 MPa, 400 MPa, 350 MPa, 300 MPa, 250 MPa, 200 MPa, 150 MPa, 100 MPa, 50 MPa, 10 MPa, or any other suitable amount less (or more) than the second stiffness. In some embodiments, the stiffness differential between the first stiffness and the second stiffness falls within a range, such as a range including any two of the foregoing stiffnesses or any other range between 1 MPa and 99.9 GPa. By way of non-limiting illustration, in some embodiments, the second stiffness is between 5 MPa and 95 MPa stiffer than the first stiffness (or vice versa). Moreover, in some embodiments, the second stiffness is between 5 MPa and 95 MPa stiffer than the first stiffness (or vice versa).

In some embodiments, by including multiple load-bearing regions (e.g., the first 102 and second 104 load-bearing regions) with varying stiffnesses, a wide variety of endplate 220 topologies 222 can be accommodated with a single implant 100. Indeed, in some embodiments, the implant causes a desirable load to be exerted across a large percentage of a surface area of an endplate (e.g., more than 50%, 60%, 70%, 80%, 90%, 95%, or 99%, or even up to 100% of the surface area of the endplate) notwithstanding variations in the topology of the endplate or variations between different endplates. In some cases, the desirable load to be exerted across the endplate is between 50 MPa and 5 GPa (or any subrange thereof). For example, in some embodiments, the implant is configured to provide a load of between 300 MPa and 1.2 GPa (±150 MPa) across a large percentage of a variety of different endplates with different topologies. Moreover, in some embodiments, the implant is configured to provide a load of between 150 MPa and 1.1 GPa (±150 MPa) across a large percentage of a variety of different endplates with different topologies.

According to some embodiments, the implant 100 is tailored to conform not only to different depths 224 of endplate 220 topology 222, but also to different regions of the endplate (e.g., the central region 226, the posterior region 228, the left region 230, the right region 232, or the anterior region 234). In this regard, endplates generally vary in stiffness, strength (e.g., load-bearing capacity), and shape across their various regions. In some embodiments, the implant is tailored to meet specific needs depending on these natural variations. For example, in some embodiments, the implant includes one or more regions that correspond with one or more regions of an endplate. In this regard, some embodiments of the implant include one or more faces (e.g., a rostral face, a caudal face, or another face) that include one or more of a central region (e.g., corresponding with the central region 226 of an endplate), a posterior region (e.g., corresponding with the posterior region 228 of an endplate), a left region (e.g., corresponding with the left region 230 of an endplate), a right region (e.g., corresponding with the right region 232 of an endplate), or an anterior region (e.g., corresponding with the anterior region 234 of an endplate).

In some embodiments, one or more regions of the implant 100 are modified to have one or more differences, such as a different stiffness, a different size, a different shape, a different contact area, a different pore density, a different material composition, different structure, or another different attribute. By way of non-limiting example, in some embodiments, at least one of a posterior region, a left region, a right region, an anterior region, or a central region of the implant has a greater (or lesser) stiffness than at least one other region of the implant. Where this stiffness is greater, this can allow a greater load to be transferred to the corresponding region of the endplate. In this regard, the central region of the bony endplate can sometimes receive the least amount of load from an implant, as the depth of the topology tends to be greatest at the central region (so the deflection of the corresponding central region of the implant is least, compared to the other regions). Thus, to compensate, some embodiments of the central region of the implant have increased stiffness (or increased length, or another suitable difference), thereby increasing the load applied to the central region of the endplate (and causing a more uniform load to be applied to the endplate overall).

In accordance with the foregoing, some embodiments of the implant 100 have a conforming region 101 (e.g., a first load-bearing region 102) that is uniformly conforming, but some embodiments of the implant have a conforming region that is adapted across various portions of the implant. In this regard, differences included in the conforming region (if any) can be arranged in any suitable manner. In some embodiments, the differences within the conforming region closely align with the regions of the bony endplate 220. In some embodiments, the conforming region includes differences within the various regions. For example, some embodiments of the conforming region include a gradient, with a particular difference (e.g., stiffness) increasing or decreasing medially to laterally, left to right, anterior to posterior, central to exterior, or in another configuration throughout all or a portion of the implant. Some embodiments include differences within the conforming region arranged in a custom-tailored manner or in another specific arrangement (e.g., in a particular geometric pattern).

In some embodiments, the implant 100 is configured to conform to a single endplate 220, but some embodiments of the implant are configured to conform to multiple endplates. For example, some embodiments of the implant include multiple faces, such as a rostral face and an opposing caudal face, a ventral face and an opposing dorsal face, and a right face with an opposing left face. In this regard, some embodiments of the implant are “double-sided”, with a rostral conforming region 101a on a rostral face of the implant (configured to conform with a caudal-facing end plate above the implant) and a caudal conforming region 101b on a caudal face of the implant (configured to conform with a rostral-facing end plate below the implant). As with the various regions (or portions of regions) within a conforming region (as discussed above), where multiple conforming regions are included, the conforming regions can differ from each other in any suitable manner (including in stiffness, size, shape, contact area, pore density, material composition, structure, or any other suitable attributes). In any case, any of the features and characteristics discussed herein can be applied to one or both of the conforming regions. As an example, in some embodiments, the rostral face includes a first load-bearing region 102 and a second load-bearing region 104, and the caudal face also includes a first load-bearing region and a second load-bearing region.

According to some embodiments, the implant 100 is configured to be “sandwiched” between two vertebrae. Accordingly, in some cases, when a rostral endplate 220a applies load to an implant endplate (e.g., a rostral conforming region 101a), the load is applied on the other side as well (e.g., at the caudal conforming region 101b loaded against a caudal endplate 220b).

Although the load-bearing regions of the implant 100 can be configured in any suitable manner to allow for conforming to an endplate as set forth above, some embodiments include a particular arrangement of the load-bearing regions. In this regard, load-bearing regions are, in some embodiments, arranged in series, in parallel, or in any combination thereof. When load-bearing regions are configured in series, exerting a load upon one region automatically causes a load to be exerted upon the other region as well. To illustrate, springs placed end-to-end (e.g., with one spring on top of the other) are arranged in series, such that pushing down on the top spring pushes down on the whole stack, and the bottom spring also compresses. Thus, when load-bearing regions are arranged in series, each of the load-bearing regions is simultaneously acted upon when loaded (although a load-bearing region with less stiffness may compress more, even though each region receives the same load). Conversely, when load-bearing regions are configured in parallel, they can (to an extent) operate independently of each other. For example, when two springs are configured in parallel (e.g., by being placed side-by-side, not touching), a load can press down on both springs at the same time (thereby exerting the same load on each spring), but a load can also press down on only one of the springs, or a heavier load can be exerted on one spring than on the other. Moreover, if one spring is longer than the other, it is possible to press down in a manner that allows only the longer spring to exert a load until the longer spring reaches a level of compression that causes it to match the height of the shorter spring, at which point the pressure can be exerted on both springs together.

As the present systems and methods relate to the foregoing, some embodiments of the implant 100 include two or more load-bearing regions (e.g., the first load-bearing region 102 and the second load-bearing region 104) arranged in series. This notwithstanding, in some embodiments, two or more load-bearing regions (e.g., the first load-bearing region and the second load-bearing region) are arranged in parallel, thereby allowing for increased conformity with a bony endplate 220 in some cases. In some embodiments, the implant includes load-bearing regions with a combination of series and parallel configurations (e.g., a rostral first load-bearing regions and a caudal first load-bearing region arranged in series, and a rostral second load-bearing region and a caudal second load-bearing region also arranged in series, where the rostral and caudal first load-bearing regions are arranged in parallel to the rostral and caudal second load-bearing regions). By way of non-limiting illustration, FIG. 16 shows an implant 100 in which the first load-bearing region 102 and the second load-bearing region 104 are formed in parallel, with the first load-bearing region including a first strand 110 and the second load-bearing region including a second strand 112 that are configured to resiliently operate independently of each other, as discussed in more detail below.

While the implant 100 can conform to one or more endplates 220 (e.g., in accordance with any of the foregoing) in any suitable manner, some embodiments, as illustrated generally in FIGS. 16-21, include one or more particular components (e.g., frames 106, porous structures 108, or other components) useful for enabling the implant to perform its intended functions.

In some embodiments, the implant includes one or more frames 106. The frame can include any component suitable for giving the implant 100 a general structure, for joining other components together, for coupling with or actuating one or more fixation mechanisms, or for providing boundaries for the implant (e.g., edge boundaries, perimeter boundaries, passage boundaries, or other boundaries). For example, some embodiments of the frame include one or more rings, flanges, solid pieces, hollow pieces, bars, braces, scaffolds, or other framing components. By way of non-limiting illustration, FIGS. 16-21 show embodiments of an implant 100 with a frame 106 that includes a rostral frame piece 106a around a perimeter of the rostral face and a caudal frame piece 106b around a perimeter of the caudal face. Additionally, some embodiments include a frame 106 with a rostral cavity frame piece 106c formed around a perimeter of a rostral opening of a cavity 114 (as shown in FIGS. 17-19), and some embodiments further have a caudal cavity frame piece on the caudal side opposite the rostral cavity frame piece (not shown).

It is important to note that the frame 106 is not limited to framing a perimeter. In some embodiments, the frame forms all or part (e.g., between 0% and 100%, or any subrange thereof, such as between any of 5%, 10%, 15%, 25%, 50%, 75%, 90%, 95%, or another percentage) of one or more faces of the implant 100 (e.g., the rostral face, the caudal face, the left face, the right face, the dorsal face, or the ventral face) or of one or more other surfaces of the implant (e.g., an interior surface of a cavity 114 or another passage). Furthermore, the frame can have any suitable dimensions for providing the general shape, size, or structure for an implant, but in some cases the frame is configured to provide the general shape, size, and structure of an intervertebral spinal implant. For example, some embodiments of the frame provide a structure for one or more edges (e.g., a perimeter) of any of the faces of the implant. Some embodiments of the frame form one or more faces.

Although the frame 106 can be included in (or in connection with) any part or parts of the implant 100 (e.g., part of a first load-bearing region, a second load-bearing region, an additional load-bearing region, or another portion of the implant entirely), the frame of some embodiments forms a part of the second load-bearing region 104. Accordingly, in some embodiments, a stiffness of the frame contributes to the stiffness of the second load-bearing region. Thus, the frame can have any stiffness suitable for contributing to the desired stiffness of the second load-bearing region (as discussed above). Indeed, any load-bearing region (such as the second load-bearing region) can derive any portion of its stiffness (anywhere from 0% to 100%, or any subrange thereof) from the frame. For example, embodiments of the second load-bearing region can derive 5%±3%, 10%±5%, 25%±10%, 50%±20, 75%±10%, 90%±5%, 95%±3%, or any other suitable percentage of its stiffness from the frame. In some embodiments, the stiffness of any particular load-bearing region derived from the frame is negligible (and is instead derived from other components, as discussed below).

Some embodiments of the various surfaces of the implant 100 (e.g., any face of the implant, the implant body (e.g., the rigid region 103), or the implant endplates (e.g., the conforming region 101)) can have any suitable characteristic that allows the implant to function as intended, including being planar, contoured, defining one or more openings, being porous, or having any other suitable characteristics. Indeed, some embodiments of the implant include one or more porous structures 108. In this regard, a properly configured porous structure can be highly beneficial in promoting healthy bone growth. Indeed, the cells generally responsible for bone growth, namely osteocytes and osteoblasts, typically work together to form bone as needed within the body. But these cells will typically only form bone under proper conditions, including when the cells experience proper loads and stresses, when a network of blood vessels is available to supply needed nutrients, and when gaps to be filled by bone are of a proper size. When proper conditions are not available, bone often cannot or will not grow. For example, when bone does not experience proper loading (e.g., due to hypoloading), it generally will not grow and can even be resorbed. Additionally, when gaps (i.e., pores) to be filled are too large, bone may not be able to bridge the gap and often will not grow. Similarly, if pores are too small, bone growth may be impeded by the insufficient size. Thus, some embodiments of the implant employ a combination of a porous structure with proper loading (e.g., as a result of the conforming region) to stimulate improved bone growth.

Where embodiments include a porous structure 108, the porous structure can include any structure having gaps, spaces, air pockets, voids, cavities, hollows, or other pores capable of promoting bone growth (e.g., cortical bone growth, trabecular bone growth, or other bone growth).

According to some embodiments, the porous structure 108 includes one or more pluralities of pores. The pores can include any gaps, spaces, voids, air pockets, cavities, hollows, or other pores that are defined by the porous structure. For example, a hole in a strand qualifies as a pore in some embodiments, and a gap between multiple strands (or multiple portions of the same strand) qualifies as a pore in some embodiments.

In some embodiments, each pore of the plurality of pores has a pore size, or the plurality of pores has an average pore size, of between 10 μm and 3,000 μm or any subrange thereof. For example, in some embodiments, the plurality of pores each have a pore size, or they have an average pore size, of between 100 μm and 1,000 μm, between 150 μm and 700 μm, between 150 μm and 650 μm, or any other subrange of between 10 μm and 3,000 μm. That said, a pore size of between approximately 150 μm and 650 μm (±100 μm) may be particularly useful for promoting bone growth in combination with the loads placed on bony endplates 20 as a result of the stiffnesses selected for the load-bearing regions of some embodiments of the implant 100 (as discussed above).

The plurality of pores can include any number of pores (e.g., between 2 and 50,000, or any subrange thereof, such as 100±50, 1000±500, 1500±750, 10,000±5000, or any other desired number of pores for promoting bone growth). The pores can also be positioned in any manner helpful for promoting bone growth, but in some embodiments, they are exposed at one or more faces of the implant 100 (e.g., any or all of the caudal face, the rostral face, the ventral face, the dorsal face, the left face, or the right face), which in some cases allows bone to access (e.g., grow into) the pores or allows for fluid to circulate within the implant.

In some embodiments, the plurality of pores is interconnected (e.g., each of the plurality of pores is fluidically interconnected with each of the other of the plurality of pores). In some embodiments, the porous structure 108 includes multiple pluralities of pores, which in some cases are interconnected with each other, and in other cases are not interconnected with each other (although the pores within a plurality may still be internally interconnected).

According to some embodiments, the pores of the porous structure 108 are defined by one or more strands. The strands can include any component or material capable of defining pores, such as one or more meshes, grids, filaments, foams, lattices, wires, grates, strings, springs, coils, webs, laces, filigrees, frames, frets, screens, reticulations, or other porous components or components that can define pores around them (whether they form strips, porous bodies, or other structures or configurations). By way of non-limiting illustration, FIGS. 16-21 show an implant 100 having a porous structure 108 formed of a plurality of strands (which, in the FIGS., are generally interwoven, interconnected, filamentous, and spring-like).

Where the porous structure 108 includes one or more strands, the strands can have any configuration suitable for forming the porous structure. For example, in some embodiments, the strands (or portions thereof) are straight, curved, spiraled, twisted, zig-zagged, accordioned, branching, randomly dispersed, geometrically arranged, interwoven, interconnected, separate, independent, uniform, crossed with other strands, woven together, parallel to each other, diagonal to each other, placed with any suitable orientation with respect to each other, varied, or otherwise configured. In some embodiments, one or more of the strands are arranged in a spring-like configuration such that a stiffness is derived from compression of the strands. According to some embodiments, a combination of the material from which the porous structure is formed and the spring-like shape of the material results in a spring constant that yields the desired stiffness of the implant 100 (or the stiffness of one or more of the implant's load bearing regions).

In embodiments in which the porous structure 108 comprises strands, the strands can also extend through any portion of the porous structure and in any orientation. For example, the strands (individually or collectively) can extend through the whole porous structure or through only a portion of the porous structure. By way of non-limiting illustration, FIGS. 16-21 illustrate an implant 100 with a first strand 110 extending from a rostral-most point at the first load-bearing region 102 on the rostral face to a caudal-most point at the first load-bearing region 102 on the caudal face; and with a second strand 112 extending from a rostral portion of the frame 106 to a caudal portion of the frame 106.

Where a strand includes a spring-like configuration, any shape can be used that results in spring-like action of the strand. For example, some embodiments of the strand include a configuration of a torsion spring, a coil spring, a conical spring, a compression spring, a helical spring, a leaf spring, a triangular spring, a rectangular spring, a diamond spring, a spring with another geometric configuration, a single-wire spring, a multi-wire spring, a spring grid, any spring as discussed in the PCT application publication WO2017100366A1 (entitled “Porous interbody spacer”) which is incorporated herein by reference in its entirety, or another type of spring. Furthermore, multiple strands operating as springs can operate dependently of each other (e.g., they are interconnected or coupled in a series configuration) or independently of each other (e.g., they are not interconnected, or they are coupled in a parallel configuration). Even where strands are not interconnected (e.g., not physically joined together), strands can be interwoven with each other, such that one strand passes through a negative space not occupied by the other strand (but within the boundaries defined by such strand) without connecting to (or, in some cases, without even contacting) the other strand. By way of non-limiting illustration, FIG. 16 shows an implant 100 with a porous structure 108 having a first strand 110 and a second strand 112. In this Figure, the first strand 110 is generally thinner (on average) than the second strand 112 (although in some embodiments, thicknesses are the same or the first strand 110 is thicker). Further, in this Figure, the first strand 110 is formed into a 3D diamond-spring lattice, being interconnected with a plurality of additional strands in a diamond pattern throughout the porous structure. The second strand 112 is also formed into a 3D diamond-spring lattice, being interconnected with another plurality of additional strands in a diamond pattern throughout the porous structure 108. However, in accordance with the embodiments shown in this Figure, the first strand 110 and the second strand 112 are not interconnected (and thereby are configured to operate in parallel, independently of one another), but they are interwoven (each occupying the negative space of the other) such that each extends throughout the porous structure 108.

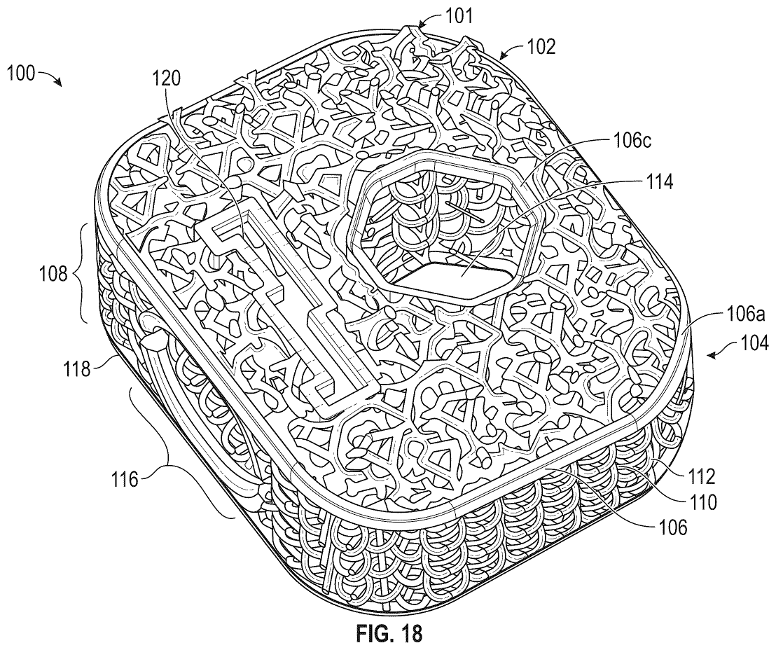

The porous structure 108 can have any number of strands, which can be part of one or more groups of strands having similar characteristics. For example, in some embodiments, the first strand is interconnected with additional strands (which, in some cases, have a similar size, shape, stiffness, or other configuration as the first strand). Similarly, in some embodiments, the second strand is interconnected with additional strands (which, in some cases, have a similar size, shape, stiffness, or other configuration as the second strand). Indeed, some embodiments have between 1 and 10,000 strands, or any subrange thereof. For example, some embodiments have between 10 and 5,000 strands, 20 and 500 strands, 50 and 200 strands, or any other number of strands. As a non-limiting illustration, FIG. 18 shows a porous structure 108 having a first strand 110 and a second strand 112, where the second strand forms a coil and where the first strand is interwoven, but not interconnected, with the second strand. However, the first strand is part of a first group of strands that are configured similarly to the first strand, and the second strand is part of a second group of strands that are configured similarly to the second strand.

In some embodiments, the configuration of the porous structure 108 at least partially gives rise to the conforming region 101. In this regard, some embodiments of the porous structure include at least part of the first load-bearing region 102 and at least part of the second load-bearing region 104. For example, in some cases, a portion of the porous structure that includes the first load-bearing region 102 extends beyond (e.g., extends farther rostrally, caudally, or both) a portion of the porous structure that includes the second load-bearing region 104. By way of non-limiting illustration, FIGS. 16-21 show embodiments of the implant 100 in which a second strand 112 of the porous structure 108 terminates at a frame 106 (and therefore does not extend rostrally or caudally past the frame), thereby forming the second load-bearing region 104, whereas the first strand 110 of the porous structure extends beyond the frame (e.g., rostrally beyond the rostral face and caudally beyond the caudal face), thereby forming the first load-bearing region 102. In some embodiments, a lower stiffness is achieved at the first load-bearing region (e.g., where the first strand extends beyond the second strand), while a greater stiffness is achieved at the second load-bearing region (e.g., where load is exerted upon both the first strand and the second strand together).

In embodiments where the first load-bearing region 102 extends past the second load-bearing region 104 (e.g., where the first strand 110 extends past the second strand 112, past the frame 106, or both), it can extend any suitable distance allowing it to place a desired load on the endplate 220. For example, some embodiments of the first load-bearing region 102 extend far enough past the second load-bearing region to place load on an endplate 220 at a portion where the topology 222 reaches a maximum depth 224. Indeed, some embodiments of the first load-bearing region extend between 0.1 mm and 10 mm (or any subrange thereof) beyond the second load-bearing region. For instance, some embodiments of the first load-bearing region extend between 1 mm and 9 mm, 2 mm and 8 mm, 3 mm and 7 mm, or any other suitable range of distances (maximally or on average) past the second load-bearing region. Additionally, as mentioned above, various portions of the load-bearing regions can differ from each other, so in some cases one strand in the first load-bearing region (e.g., a first strand 110) can extend beyond not only a strand in the second load-bearing region (e.g., a second strand 112), but also beyond another strand in the first load-bearing region (e.g., a third strand). For example, in some embodiments, a portion of a load-bearing region (e.g., one or more particular strands of the load-bearing region) is longer (or higher, or extended farther past another load-bearing region) than another portion of the load bearing region (such as at an anterior portion, a posterior portion, a right portion, a left portion, a central portion, or any other suitable portion).

In some embodiments, the conforming region 101 (e.g., the first load-bearing region 102) includes one or more contact surfaces 128 where the conforming region is configured to contact the endplate 220. Such contact can be either direct (e.g., where a portion of the conforming region, such as a first strand 110 makes direct contact with the bone of the endplate) or indirect (e.g., where one or more intervening components-such as a covering, a coating, a dressing, a bead, a sheet, or another intervening component-makes contact with the endplate 220).

Some embodiments of the conforming region 101 include one or more adaptations, with such adaptations including anything allowing the contact between the conforming region and the implant to be safer, more comfortable, more effective at applying a desirable load, more effective at stimulating bone growth, or otherwise more beneficial to the patient or to the implant. For example, some embodiments of the implant 100 have increased surface area at a contact surface. By way of non-limiting illustration, FIG. 21 shows a porous structure 108 having a first strand 110 and a second strand 112. The second strand terminates at a frame 106, forming a second load-bearing region. The first strand extends beyond the frame, forming a first load-bearing region. While the majority of the first strand is, in some embodiments, thinner than the second strand (e.g., the cross-sectional diameter of the first strand is smaller than the cross-sectional diameter of the second strand), the first strand includes a transition portion 126 in which the cross section becomes thicker, thereby providing increased surface area at a point of contact 128. In some such embodiments, this configuration provides improved strength to the exposed portion of the strand (e.g., the portion of the first strand extending beyond the frame), thus reducing non-axial loading (e.g., bending, shear, lateral movement, or other loading that does not result in axial compression of the spring-like porous structure).

According to some embodiments, the implant 100 includes one or more passages. The passage can include any cavities, receptacles, slots, recesses, voids, notches, grooves, holes, bores, depressions, orifices, or other passages. In some embodiments, the passage is free of, or defined by, the porous structure 108 or another component of the implant. The passage can be formed in or through any portion of the implant, such as in a single face (e.g., in the ventral face), from any one face to another (e.g., from the rostral face to the caudal face), or internally (e.g., without having an opening in any face). The passage can also have any suitable dimensions, such as by accounting for anywhere between 1% and 50% of the volume of the implant (or any subrange thereof, such as 10%±5%, 15%±7%, 25%±12%, or any other subrange of the implant's volume).

According to some embodiments having a passage, the passage includes a cavity 114. While this cavity can perform any suitable function, in some embodiments it receives one or more bone graft materials, such as morcellized bone (living bone), thereby providing a seed for initiating bone growth into the implant 100 after implantation. In some embodiments, the cavity extends only partway through the implant, but some embodiments of the cavity extend from one face of the implant to another. The cavity can also be positioned on any portion of the implant, at any angle, as might be useful for providing bone graft materials. The cavity can further have any suitable shape for receiving bone graft materials. By way of non-limiting illustration, FIGS. 17-19 show a cavity 114 formed from the rostral face to the caudal face of the implant 100, extending through an entirety of the implant, generally centered between the right and left faces, and slightly off-center toward the dorsal face between the ventral and dorsal faces.

According to some embodiments having a passage, the passage includes one or more elements of a primary fixation mechanism 116 (as discussed in more detail below), such as a receptacle 118 or a slot 120. By way of non-limiting illustration, FIGS. 17-20 show an implant 100 with passages including one or more slots 120 configured to house one or more anchors 124 of a primary fixation mechanism 116 and to permit the anchor to travel through the slot to be embedded in a portion of the endplate 220 to thereby fix the implant 100 in place. The passages of FIGS. 17-20 also include one or more receptacles 118 for housing an actuation mechanism 122 of the primary fixation mechanism 116, which actuation mechanism can be actuated (e.g., rotated, switched, slid, toggled, flipped, translated, pressed, or otherwise actuated) in order to deploy or disengage the anchors.

Where the implant 100 includes a primary fixation mechanism 116, the primary fixation mechanism can include any suitable mechanism for fixing the implant in place (e.g., by anchoring it to one or more endplates 220). Indeed, the primary fixation mechanism can include one or more blades, spikes, hooks, teeth, or other anchors, along with any suitable actuator (whether operating through rotation, transverse movement, leverage, or through any other manner) to deploy such anchors (where deployment is necessary).

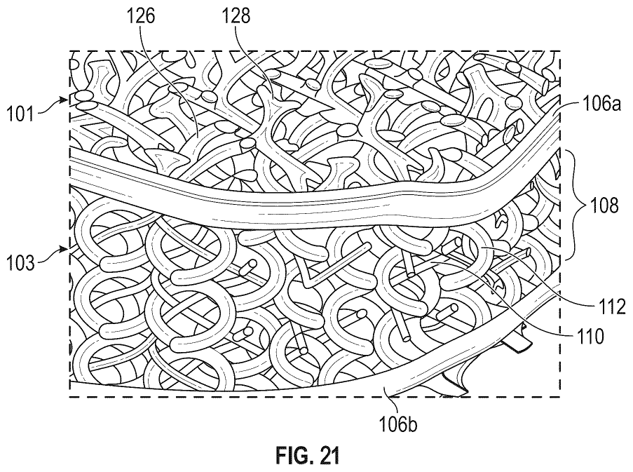

The anchors 124 of the primary fixation mechanism 116 can be configured to deploy in any suitable manner. For example, some embodiments of the primary fixation mechanism are configured to deploy anchors directly outward along the rostral/caudal axis (e.g., orthogonal to the rostral and caudal faces of the implant 100, or orthogonal to the endplates 220). By way of non-limiting example, FIG. 20 shows an implant 100 with a primary fixation mechanism 116 configured to deploy anchors 124 (comprising blades) at an approximately 0° (±10°) angle.

The implant 100 can be formed in any suitable manner, such as by casting (e.g., continuous casting, die casting, mold casting, resin casting, sand casting, or other casting), molding (e.g., metallurgy, compaction, injection molding, or other molding), forming (e.g., forging, extrusion, pressing, or other forming), machining (e.g., milling, turning, drilling, or other machining), joining (e.g., welding or other joining), additive manufacturing (e.g., 3D printing, deposition, layered manufacturing, or other additive manufacturing), or otherwise. For example, some embodiments are formed through additive manufacturing. Accordingly, some embodiments are specifically configured to be easily formed through 3D-printing or other additive manufacturing techniques.

The described systems and methods can be modified in any suitable manner. For example, although some embodiments of the implant 100 are specifically configured to be used in spinal surgery, some embodiments are configured to be used in other parts of the body (e.g., in joint replacements, bone grafts, or other types of surgery). Accordingly, some embodiments of the implant are not limited to being spinal implants.

As mentioned above, some embodiments of the implant 100 have more load-bearing regions than just a first load-bearing region 102 and a second load-bearing region 104. In this regard, in some embodiments, the conforming region 101 includes a first load-bearing region and the rigid region 103 includes a second load-bearing region, but at least one of the conforming region or the rigid region also includes a third load-bearing region. A fourth load bearing region, a fifth load-bearing region, a sixth load-bearing region, or any other number of additional load-bearing regions is also included, in some embodiments. The various load bearing regions can be arranged in parallel or series, and may be positioned in any manner with respect to each other. For example, in some embodiments a third load-bearing region is positioned to surround the first load-bearing region, be positioned next to the first load-bearing region, be placed in a different portion of the implant than the first load-bearing region, extend past the first load-bearing region, or be otherwise positioned with respect to the first load-bearing region.

In some embodiments, one or more of the rigid region 103 or the conforming region 101 have multiple layers of differing stiffness, whether in layers or stratified. For example, in some embodiments, the conforming region is configured to become progressively more stiff as it approaches the rigid region. In some embodiments, the whole implant 100 (or any portion thereof) is stratified, with a spectrum of stiffness on one or more faces. In some embodiments, this effect is created using multiple load bearing regions (e.g., as set forth in the previous paragraph), and in some embodiments, this effect is created using stiffness variations within one or more load-bearing regions. Accordingly, in some embodiments, a single load-bearing region includes multiple stiffnesses, such as any of the stiffnesses discussed herein.

Another suitable modification relates to custom-tailoring the implant 100 for specific patients. In particular, in some embodiments, a scan (e.g., an X-ray, a CT Scan, an MRI, an ultrasound, or another type of imaging) is performed and then the implant is created (or modified) based on one or more characteristics of the scan. For example, in some embodiments the size, stiffness, shape, pore size, or any other suitable attribute is initialized or adjusted based on one or more attributes of the patient as recorded during the scan.

As another potential modification, in some embodiments, the first load-bearing region 102 and the second load-bearing region 104 are integrally formed. In some embodiments, a transition between stiffnesses is abrupt, and in some embodiments, there is a gradient of stiffness transition.

Although this disclosure generally discusses implants 100 having a conforming region 101 generally, or a rostral conforming region 101a and a caudal conforming region 101b, it is important to note that the implant can have any number of conforming regions (e.g., a first conforming region, a second conforming region, a third conforming region, a fourth conforming region, and any number of additional conforming regions). In some embodiments, the conforming regions align with one or more load-bearing regions, but in some embodiments, multiple load-bearing regions form a single conforming region, and in some embodiments, multiple conforming regions are formed from a single load-bearing region.

In addition to the aforementioned features, the described systems and methods can include any other suitable feature. For example, additional features can be added to the conforming region 101 due to the conforming region's unique property (in some embodiments) of extending past the rostral and caudal faces of the rigid region 103 (or body) of the implant 100, thereby resulting in conforming artificial implant endplates that can complement a wide variety of natural endplates 20. Some such additional features include one or more individual anchors (to anchor the implant in place even more thoroughly than a primary fixation mechanism 116 on its own); protein coatings or other use of biological materials to stimulate bone growth; or individual trimmings, extensions, or other adjustments of single strands in order to accommodate specific aberrations in endplate topology 222.

Any and all of the components in the figures, embodiments, implementations, instances, cases, methods, applications, iterations, and other parts of this disclosure can be combined in any suitable manner. Additionally, any component can be removed, separated from other components, modified with or without modification of like components, or otherwise altered together or separately from anything else disclosed herein.

As used herein, the singular forms “a”, “an”, “the” and other singular references include plural referents, and plural references include the singular, unless the context clearly dictates otherwise. For example, reference to a fastener includes reference to one or more fasteners, and reference to actuators includes reference to one or more actuators. In addition, where reference is made to a list of elements (e.g., elements a, b, and c), such reference is intended to include any one of the listed elements by itself, any combination of less than all of the listed elements, and/or a combination of all of the listed elements. Moreover, the term “or” by itself is not exclusive (and therefore may be interpreted to mean “and/or”) unless the context clearly dictates otherwise. Similarly, the term “and” by itself is not exclusive (and therefore may be interpreted to mean “and/or”) unless the context clearly dictates otherwise. Furthermore, the terms “including”, “having”, “such as”, “for example”, “e.g.”, and any similar terms are not intended to limit the disclosure, and may be interpreted as being followed by the words “without limitation”.

In addition, as the terms “on”, “disposed on”, “attached to”, “connected to”, “coupled to”, etc. are used herein, one object (e.g., a material, element, structure, member, etc.) can be on, disposed on, attached to, connected to, or otherwise coupled to another object-regardless of whether the one object is directly on, attached, connected, or coupled to the other object, or whether there are one or more intervening objects between the one object and the other object. Also, directions (e.g., “front”, “back”, “on top of”, “below”, “above”, “top”, “bottom”, “side”, “up”, “down”, “under”, “over”, “upper”, “lower”, “lateral”, “right-side”, “left-side”, “base”, etc.), if provided, are relative and provided solely by way of example and for ease of illustration and discussion and not by way of limitation.

The described systems and methods may be embodied in other specific forms without departing from their spirit or essential characteristics. The described embodiments, examples, and illustrations are to be considered in all respects only as illustrative and not restrictive. The scope of the described systems and methods is, therefore, indicated by the appended claims rather than by the foregoing description. All changes that come within the meaning and range of equivalency of the claims are to be embraced within their scope. Moreover, any component and characteristic from any embodiments, examples, and illustrations set forth herein can be combined in any suitable manner with any other components or characteristics from one or more other embodiments, examples, and illustrations described herein.

What is claimed is:

1. A spinal implant comprising:
a first load-bearing region having a first stiffness; and
a second load-bearing region having a second stiffness,
wherein the first stiffness is different than the second stiffness.

2. The implant of claim 1, wherein the first load-bearing region and the second load-bearing region are configured to be loaded in series.

3. The implant of claim 1, wherein the first load-bearing region and the second load-bearing region are configured to be loaded in parallel.

4. The implant of claim 1, wherein the first load-bearing region is configured to be positioned closer to a point of contact than the second load-bearing region.

5. The implant of claim 1, wherein the first load-bearing region is configured to conform to an uneven surface.

6. The implant of claim 1, wherein a combination of the first load-bearing region with the second load-bearing region provides a more uniform stiffness across a variety of contact surfaces than an implant having only one load bearing region of a single stiffness.

7. The implant of claim 1, further comprising a central region having a third stiffness, wherein the third stiffness is different than the first stiffness.

8. The implant of claim 7, further comprising a porous structure.

9. A spinal implant comprising:
an implant body;
a first conforming region coupled to the implant body and configured to be disposed adjacent to a first natural endplate of a first spinal segment of a patient, wherein the first conforming region comprises:
a first contact surface configured to contact at least a portion of the first natural endplate; and
a first load-bearing region having a first stiffness;
a second load-bearing region having a second stiffness that is greater than the first stiffness,
wherein the first contact surface is coupled to the first load-bearing region such that a shape of the first contact surface is configured to conform to a shape of the first natural endplate.

10. The spinal implant of claim 9, wherein the first load-bearing region comprises the first contact surface.

11. The spinal implant of claim 9, further comprising a second conforming region coupled to the implant body opposite the first conforming region, the second conforming region being configured to be disposed adjacent to a second natural endplate of a second spinal segment of the patient, wherein the second conforming region comprises:
a second contact surface configured to contact at least a portion of the second natural endplate; and
a third load-bearing region having a third stiffness that is less than the second stiffness.

12. The spinal implant of claim 11, wherein the third stiffness is approximately equal to the first stiffness.

13. The spinal implant of claim 11, further comprising a porous structure extending through the implant body.

14. The spinal implant of claim 13, wherein the porous structure comprises at least a portion of the first load-bearing region and at least a portion of the third load-bearing region.

15. The spinal implant of claim 11, further comprising a frame at least partially surrounding the implant body.

16. The spinal implant of claim 15, wherein the frame comprises at least a portion of the second load-bearing region.

17. The spinal implant of claim 9, wherein the first load-bearing region comprises:
a central region having a central region stiffness;
a lateral region having a lateral region stiffness;
an anterior region having an anterior region stiffness; and
a posterior region having a posterior region stiffness,
wherein at least one of the central region stiffness, the lateral region stiffness, the anterior region stiffness, and the posterior region stiffness is different from another of the central region stiffness, the lateral region stiffness, the anterior region stiffness, and the posterior region stiffness.

18. A method of providing a spinal implant, the method comprising:
obtaining a spinal implant comprising:
an implant body; and
a first conforming region coupled to the implant body and configured to be disposed adjacent to a first natural endplate of a first spinal segment of a patient, wherein the first conforming region comprises:
a first contact surface configured to contact at least a portion of the first natural endplate;
a first load-bearing region having a first stiffness; and
a second load-bearing region having a second stiffness that is greater than the first stiffness,
wherein the first contact surface is coupled to the first load-bearing region such that a shape of the first contact surface is configured to conform to a shape of the first natural endplate; and
disposing the first contact surface of the first conforming region of the spinal implant adjacent to the first natural endplate of the first spinal segment of the patient in a manner that allows the shape of the first contact surface to conform to the shape of the first natural endplate.

19. The method of claim 18, further comprising forming the spinal implant via additive manufacturing.

20. The method of claim 18, wherein the spinal implant further comprises a porous structure, and wherein the method further comprises placing the porous structure in contact with the first natural endplate to promote bone growth.
